(19) United States
(12) Patent Application Publication
(10) Pub. No.: US 2016/0031937 A1
(43) Pub. Date: Feb. 4, 2016

(54) NEURAL REGENERATION PEPTIDES AND USES THEREFOR

(71) Applicant: CURONZ HOLDINGS COMPANY LIMITED, Grafton, Auckland (NZ)

(72) Inventor: Frank SIEG, Wellsford (NZ)

(21) Appl. No.: 14/773,428

(22) PCT Filed: Mar. 7, 2014
(86) PCT No.: PCT/NZ2014/000029

§ 371 Date: Sep. 8, 2015

(30) Foreign Application Priority Data
Mar. 8, 2013 (NZ) 608027

(51) Int. Cl. C07K 7/06

(52) U.S. Cl. CPC C07K 7/06; A61K 38/00

(57) ABSTRACT

This invention relates to neural regeneration peptides (NRPs), including NRP-2945, NRP-2983 and NNZ-4921, as well as the receptors that have been newly identified as interacting with these NRPs, such as CXCR4 in collaboration with CCR3. The invention further relates to methods of using these NRPs and its respective chemokine receptors, as well as compositions comprising such components.

### FIELD OF THE INVENTION

This invention relates to neural regeneration peptides (NRPs), including NRP2945, NRP 2983 and NNZ-4921, as well as the receptors that have been newly identified as interacting with these NRPs, such as CXCR4 in collaboration with CCR3. The invention further relates to methods of using these NRPs and its respective chemokine receptors, as well as compositions comprising such components.

### BACKGROUND OF THE INVENTION

NRPs

The peptides disclosed herein belong to a newly discovered peptide family, named neuronal regeneration peptides (NRPs). They are small peptides that exert an array of biological functions crucial for neuronal regeneration and are involved in promoting neuronal survival, proliferation, migration and differentiation (Gorba et al., 2006; Sieg & Antonic, 2007).

NRPs were discovered using an ex vivo rat brain slice cultivation model to screen for novel factors that induce neuronal migration. In cultivated organotypic slices of rat neocortex and dorsal thalamus (organs of the brain), a highly purified peptide (subsequently designated “Neural Regeneration Peptide” or NRP) stimulated the proliferation and migration of neurons and the formation of thalamocortical cell bridges of neuronal origin, while demonstrating extraordinary activity in protecting brain tissue from oxidative and mechanical stress. (Landgraf et al., 2005).

There are various NRP gene family members encoded within vertebrate genomes including fish, amphibians, birds, as well as mouse, rat, dog and human genomes. Full length annotated and EST-derived Nrp gene sequences only display 35-40% sequence similarity. Nevertheless, bioactive NRP sequences (11-25 amino acids in length) derived from these gene products display 55-90% sequence similarity when compared amongst the various genomes.

NRP2945 is a synthetic 11-mer peptidomimetic that has been optimized for stability and pharmacokinetics. The NRP2945 sequence Gly Arg Arg Ala Ala Pro Gly Arg Aib Gly Gly (SEQ ID NO:1) shows 80-90% sequence similarity to various NRP-related sequences. NRP2945 is very closely related to NNZ-4921, which has the sequence Gly Arg Arg Ala Ala Pro Gly Arg Ala Gly Gly (SEQ ID NO:2). NNZ-4921 is representing the naturally found sequence within the N-terminal sequence of calcium-dependent activator protein for secretion isoform 2 (CAPS-2) comprising amino acid positions 40-50. There is one difference in regard to position 43 where in CAPS-2 aspartic acid is present while this has been changed to alanine in NNZ-4921. CAPS-2 represents one of three isoforms of CAPS and it is required for calcium regulated exocytosis of secretory vesicles (Speidel et al., 2003).

NRP2983 is a synthetic 11-mer peptidomimetic GRRAAPGR-f3-Ala-GG (SEQ ID No:9) and is closely related to NRP2945 and NRP4921.

Human CAPS-2 is expressed in vesicles on the presynaptic terminals. Vesicles enriched with CAPS-2 also contain NT3 and BDNF, and thus it is believed that CAPS-2 could be involved in neuroprotection (Sadakata et al., 2004). Studies performed on embryonic and postnatal tissue show that NRP2945 is involved in survival, proliferation, migration, and differentiation. In particular, the peptide is believed to act as a chemoattractant involved in promoting cell survival during oxidative and excitotoxic stress (Gorba et al., 2006)

CCR3 and CXCR4

CC chemokine receptors (also called beta chemokine receptors) are integral membrane proteins that specifically bind and respond to cytokines of the CC chemokine family. They represent one subfamily of chemokine receptors, which belong to the larger family of G protein-linked receptors. CCR3 is a receptor for multiple inflammatory/inducible chemokines, including eotaxin (CCL11), eotaxin-3 (CCL26), MCP-3 (CCL7), MCP-4 (CCL13), and RANTES (CCL5) (Dougherty et al., 1996; Ponath et al., 1996; Youn et al., 1997; Kitaura et al., 1996; Kitaura et al., 1999; Pan et al., 2000; White et al., 1997).

CCR3 is highly expressed in both eosinophils and basophils, and is also expressed in Thl and Th2 cells and airway epithelial cells. CCR3 is believed to contribute to the accumulation and activation of eosinophils and other inflammatory cells involved in allergic responses, and may also be found at sites of parasitic infection. In addition, it is known to be a co-receptor for entry of human immunodeficiency virus, HIV-1 (Nedellec et al. 2009).

CXC chemokine receptor 4 (CXCR4) is a G-protein-coupled chemokine receptor (GPCR). It is widely expressed in leukocytes such as T-cells, B-cells, and monocytes as well as in various CNS areas (e.g., occipital, temporal cortex and spinal cord—Sehgal et al., 1998) and PNS tissues (e.g., dorsal root ganglion—Oh et al., 2001) and in various ontogenetically developing organs like lung, heart, liver, kidney, spleen, testes as well as uterus tissue during early placentation events (Singh et al., 2010). Genetically created “Knock-out” mice mutants of CXCR4 are lethal during embryonic development highlighting the importance of this chemokine receptor for overall cell survival and cellular differentiation.

There exists the paradox for the gene expression of the CXCR4 receptor system that agents like granulocyte colony stimulating factor (G-CSF) or Interleukin 21 (IL-21) that down-regulate CXCR4 gene expression under normoxic conditions have beneficial effects on cell survival and regeneration (Gupta et al., 1999; Yoshida et al., 2011). Transient up-regulation of the CXCR4 message by ciliary neurotrophic factor (CNTF) takes place during initiation of neuronal differentiation of neural precursor cells (Yang et al., 2012) followed by a sharp drop in expression thereafter. Moreover, long-term up-regulated CXCR4 gene expression is detrimental and can be observed in hyperplastic (Darash-Yahana et al., 2004) and cancerous cells.

Prior to our studies, the only known natural ligand for the CXCR4 receptor was designated as stromal cell derived factor 1 (SDF-1). Under physiological conditions, SDF-1 is secreted by bone marrow stromal cells for expansion and development of precursor B-cells. High concentrations of SDF-1 are present at inflammatory sites, so the migration of CXCR4-expressing stem cells toward an SDF-1 gradient promotes repair of various injured tissues. Furthermore, within the CNS, the ligand SDF-1 is secreted by astrocytes and neurons influencing developmental aspects of interneuronal survival, migration, and final differentiation.

There have been many reports on the pathology of CXCR4-related cancer, including CXCR4 overexpression and organ-specific metastasis among various types of cancer cells. During metastasis, SDF-1 expressed in secondary lesions functions as a chemoattractant for directional migration of CXCR4-expressing malignant cells. Approximately, 75% of all cancers show signs of dysregulated CXCR4 gene and protein expression, which qualifies CXCR4 as an important therapeutic target in cancer.

CXCR4 is one of several chemokine receptors that are up-regulated in patients with heart failure (Aukrust et al., 1998; Damas et al., 2000; Damas et al., 2001). In addition, CXCR4 has been identified as a major co-receptor that facilitates the entry of T-cell line tropic human immunodeficiency virus type 1 (HIV-1) into target host cells. The inhibitory effect of SDF-1 on HIV infection is thought to be by competitive binding to CXCR4 as well as CXCR4 down-regulation. CXCR4 is a promising molecular target for potential anti-metastatic agents and anti-HIV agents, so several CXCR4 ligands (antagonists) have been developed.

During physiological (normoxic) postnatal human brain development, CXCR4 is most prominently expressed in hippocampus and cerebellum (Van der Meer et al., 2001). However, during brain manipulation by using the technique of Mid cerebral artery occlusion (MCAO), neuronal and reactive astrocytic CXCR4 gene expression is 2-6 times up-regulated above normoxic control levels within the ipsilateral side, particularly within layer VI of the cingulated cortex (Stumm et al., 2002). On the other hand, the CXCR4-specific ligand SDF-1 is simultaneously down-regulated over several hours (Stumm et al., 2002). With this opposed expression levels of the CXCR4/SDF-1 receptor ligand system desensitization is prevented at the receptor level to allow for subsequent regeneration near the prenumbra of the MCAO-lesioned brain.

The down-regulation of the CXCR4 receptor protein is initiated by phosphorylation of its cytoplasmic tail, which is followed by the binding of 13-arrestin in which phosphorylated serine residues and a dileucine motif at the CXCR4 associated C-terminus have critical roles. The complex is sorted into late endosomes/lysosomes for the degradation pathway or for recycling endosomes. Down-regulation of CXCR4 could also occur through the stimulation of other GPCRs. The activation process of CXCR4 by SDF-1 has been well documented. Following binding of its ligand, CXCR4 undergoes dimerization and activates Gi G-proteins. However, downstream activation through CXCR4 could also occur through other G-proteins and non-G-proteins.

Upon SDF-1 binding, CXCR4 evokes downstream signalling via dissociation of heterotrimeric G proteins, followed by a decrease in intracellular cyclic adenosine monophosphate (cAMP) concentrations, up-regulation of intracellular Ca²⁺ release, and increase in extracellular-signal-regulated kinase (ERK) 1/2 phosphorylation. Another control mechanism for CXCR4 related signalling is mediated by the density of CXCR4 receptors on the plasma membrane. The actual amount of CXCR4 protein is regulated by ubiquitination/de-ubiquitination events of the receptor involving the intracellular proteasome pathway (Mines et al., 2009).

In addition to CXCR4 forming homodimers, there is evidence for CXCR4 heterodimer formation, which can lead to alternative G protein coupling besides Gi. Contento et al. provide evidence to suggest that CXCR4 and CCR5 recruitment to the immunological synapses (IS) of T cells, and subsequent receptor association, promote chemokine-induced co-stimulation of T cells. Interestingly, CXCR4/CCR5 heterodimers were shown to couple to Gq and/or G11 and generate stimulatory signals that can enhance T cell activation, thus providing a mechanism for modulating T cell behaviour (Contento et al., 2008).

Apart from involvement in bone marrow stem cell homing and effects on various immune cell types, SDF-1α/CXCR4 signalling has been shown to be a critical component of islet genesis (see, e.g., Ayse et al., 2012). The CXCR4/SDF-1 system has also been shown to be involved in neuronal chemoattraction during embryonic brain development and is also crucial for the facilitation of neuronal survival following oxidative/excitotoxic stress of brain tissue.

In the CNS, the CXCR4 ligand SDF-1 displays a variety of biological activities such as enhancing proliferation, migration, and survival of neurons and glia. The potential mechanism of SDF-1 bioactivity involves the downstream activation of extracellular regulated kinase 1/2 (ERK1/2) pathway by triggering the increase in intracellular calcium concentration (Pearson et al 2001). Furthermore, CXCR4 activation facilitates the translocation of phosphorylated beta-catenin to the nucleus, which initiates gene expression patterns favouring neuronal survival- and proliferation-promoting genes within neuronal precursor cells (Luo et al., 2006).

Notably, SDF-1 binds to a homodimeric formation of the CXCR4 receptor but shows low potency in agonizing the chemokine receptor. Concentrations in the lower nanomolar range are required, and 9 nM of SDF-1 is the minimal concentration required to chemoattract neuronal stem cells (Xu and Heilshorn, 2012). Yet, this minimal necessary concentration of SDF-1 is unlikely to be present in vivo if analysed within the sensitive period of brain development.

Moreover, it is undesirable that SDF-1 attracts immune cells that have CXCR4 receptors on the cell surface. This activity can be problematic if SDF-1 is administered after brain injury to the respective lesion site. Because of these considerations, drug development efforts in the CXCR4/SDF-1 neuroregeneration arena (see, e.g., (Ratajczak and Kim, 2012)) would greatly benefit from the discovery of receptor agonists that are more potent and have fewer negative effects on immune cells.

### BRIEF DESCRIPTION OF THE DRAWINGS

The invention will now be described by example only with reference to the figures where:

FIG. 1. The effects of NRP2945 on H₂O₂ induced cell death. Symbols *, **, ## represent statistically significant p values as described in Example 18.

FIG. 2. The effects of NRP2945 on oxygen glucose deprivation (OGD) induced cell death. Symbols *, **, ## represent statistically significant p values as described in Example 18.

FIG. 3. Time dependent effects of NRP2945 on H₂O₂ induced cell death. Symbols *, **, ## represent statistically significant p values as described in Example 18.

FIG. 4. Time dependent effects of NRP2945 on OGD induced cell death. Symbols *, **, ## represent statistically significant p values as described in Example 18.

FIG. 5. The effects of NRP2945 on oxygen glucose deprivation (OGD) induced cell death are abolished by simultaneous administration of CXCR4 inhibitor AMD3100. Symbol ## represents statistically significant p values as described in Example 18.

FIG. 6. Dependency of the survival of cerebellar granule cells on CXCR4 and CCR3 assembly and subsequent co-activation by NNZ-4921.

FIG. 7. Fold change in CXCR4 gene expression within human differentiated ESCs after NRP2945 contact relative to the untreated hESC control and compared to a human tissue cDNA library.

FIG. 8. Gene expression profile of NRP 13q13.2 after oxidative stress and in the presence of NRP2945.

FIG. 9. Chemorepulsion of DU-145 prostate cancer cells as facilitated by NRP2945.

FIG. 10. Human chromosome 13q13.2 NRP coding sequence. The forward primer (SEQ ID NO:10) is indicated in bold/ underline. The reverse primer (SEQ ID NO:11) is in reverse complement direction and indicated in bold/italics/underline.

FIG. 11. The peptidomimetic NRP2983 (SEQ ID NO:9) revealed comparative survival-promoting activities to NRP 2945. Addition of 1fM of NRP2983 to oxidatively stressed cerebellar microexplants resulted in 70% promotion of survival of cultivated cerebellar cells.

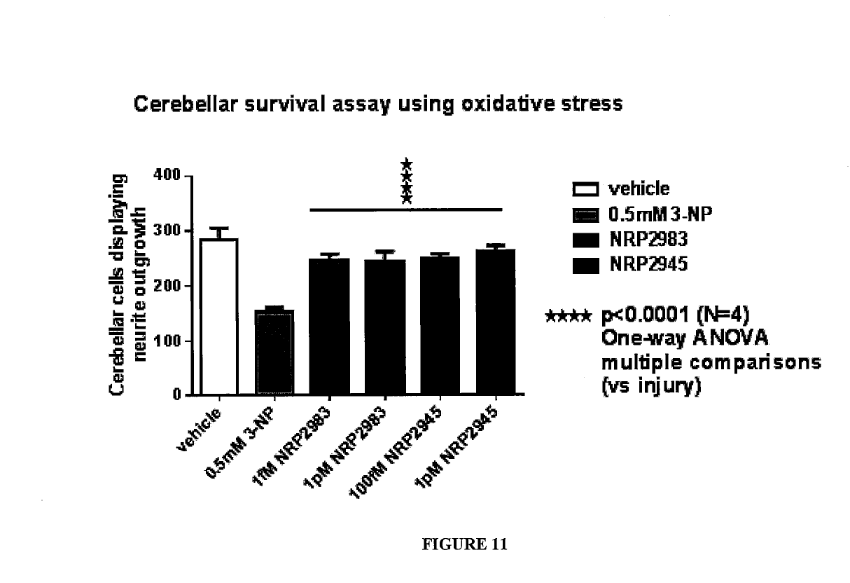

### DETAILED DESCRIPTION OF THE INVENTION

Neural regeneration peptides (NRPs) are a class of peptides that have been shown to exhibit properties desirable for promoting neural function in mammals. These functions include neural survival, neural proliferation, neuronal outgrowth, neural migration, and neuronal differentiation. Several NRPs have been previously described, and include those disclosed in U.S. patent application Ser. Nos. 10/225,838 and 10/976,699, U.S. Pat. Nos. 7,563,862, 7,767,786, 8,138,304, and 8,309,684, PCT/US02/026782, PCT/US2004/036203, PCT/1JS2006/017534, PCT/US2006/026994, and PCT/US2008/011951. Each of the above patents and patent applications is expressly incorporated herein fully by reference as if individually so incorporated.

Exemplary NRPs include the following.

(NRP2945; SEQ ID NO: 1)
GRRAAPGR-Aib-GG
(NNZ-4921; SEQ ID NO: 2)
GRRAAPGRAGG
(SEQ ID NO: 5)
GRRA-Aib-PGRAGG
(SEQ ID NO: 6)
GRRAAPGRANG
(SEQ ID NO: 7)
GRDRAAPGRAGG
(SEQ ID NO: 8)
REGRRDAPGRAGG
(NRP 2983)
(SEQ ID NO: 9)
GRRAAPGR-β-Ala-GG

As previously noted, CXCR4 receptor is a chemokine receptor of the GPCR type that is involved in trafficking of leukocytes, enzyme secretion and T-cell activation during inflammation. In the past decade, it has been found that CXCR4 is widely expressed in the CNS. Moreover, it has been shown that CXCR4 is crucially important for facilitating the migration of interneurons in the neocortex (Stumm et al., 2007). As noted above, CXCR4 has also been implicated in cancer, hyperplasia, and metastasis. In addition, CXCR4 has been identified as a major co-receptor for the entry of HIV-1 into target host cells.

GPCRs are known for their promiscuous nature apart from CXCR4. However, we have found that CXCR4 is also promiscuous. In addition to SDF-1, other high affinity ligands of CXCR4 include neural regeneration peptides, NRP2945, NRP2983 and NNZ-4921, as demonstrated herein. Notably, SDF-1 has EC₅₀ values for chemoattraction in the lower nanomolar range, while preliminary experiments show that NRP2945 has EC₅₀ values in the lower femtomolar range. Thus, the neuronal chemoattractive potency of NRP2945 may be more than 1 million times greater than that of SDF-1.

Various mechanisms of action for NRPs have been revealed by our studies, both previously and as detailed herein. Our data provided herein demonstrates that NRPs such as NRP2945 and NNZ-4921, are involved in the agonizing action (or activation) of the CXCR4 receptor. We have found that activation of CXCR4 by NRP ligands, like NRP2945 and NNZ-4921, plays a major role in preventing neuronal cell death under oxidative stress conditions. Furthermore, our current findings suggest that neuronal migration and promotion of final neuronal differentiation (see, e.g., Gorba et al., 2006) may be influenced by the interaction of NRPs with their receptor CXCR4.

We have previously determined that the ligand NRP2945 enhances human NRP gene expression in an autocrine fashion. As already reported, human chromosome chromatin bands 15q12 and 13q13.2 contain NRP gene sequences. Human embryonic W9 stem cells as well as the human carcinoma derived cell line NTERA-2 show stimulated endogenous NRP gene expression within 5-10 minutes after 100 fM and 100 pM of NRP2945 administration, respectively.

We have published also that NRPs can exert their downstream signalling by activation of ERK 1/2 phosphorylation and by the activation of the phosphatidylinositol 3-kinases (PI3K) pathway via Akt-1 phosphorylation (Gorba et al., 2006). Moreover, we show herein that the interaction between the NRP and CXCR4 can be blocked by administration of the specific antagonist AMD3100. As a highly specific antagonist, AMD3100 also blocks the binding pocket of SDF-1 when associated with CXCR4 (Liang et al., 2012).

However, in contrast to the SDF-1 interaction that requires a homodimeric configuration of the CXCR4 receptor, NRP2945 and NNZ-4921 do not bind to the homodimeric CXCR4 receptor. This was determined by radioactive ¹²⁵I-SDF-1 displacement studies using CXCR4 homodimeric receptors and unlabelled NNZ-4921 and NRP2945 in competition. The NRP molecule is not able to displace the radioactively labelled SDF-1 molecule from the homodimeric CXCR4 receptor complex (¹²⁵I-SDF-1 binding study using NNZ-4921 as competitive ligand used within recombinant homodimeric CXCR4 expressing HEK293 cells. NNZ-4921 was not able to displace the radioactive ligand SDF-1—conducted by CEREP, France).

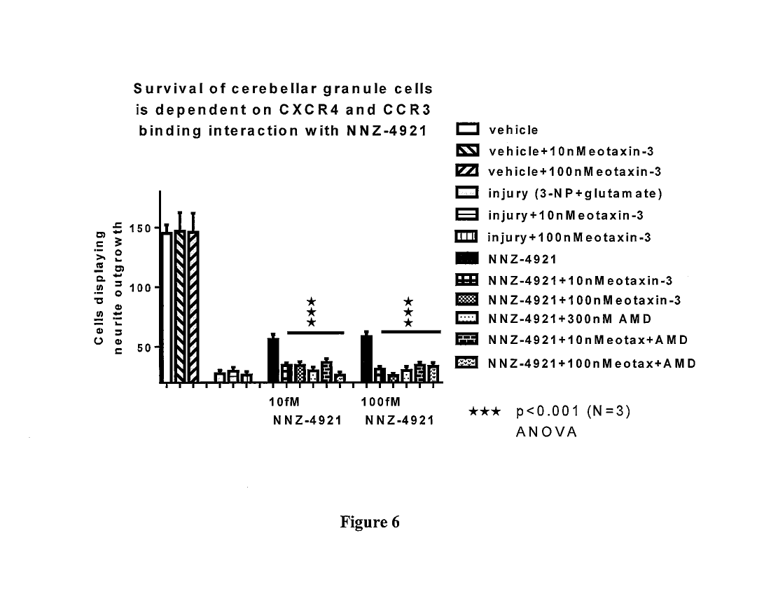

Furthermore, we demonstrate herein that NRP2945 and NNZ-4921 interact with a heterodimeric configuration of CXCR4 and another chemokine receptor, named CCR3. This was elucidated by addition of a ligand of the CCR3 receptor (eotaxin-3 or CCL-26) to oxidatively stressed cerebellar microexplants in the presence of NNZ-4921 (see FIG. 6, herein). The survival-promoting activity of NNZ-4921 was completely blocked by the partial agonist eotaxin-3. When eotaxin-3 was administered to cerebellar microexplants without addition of NNZ-4921, this had no effect on the overall neuronal survival rate neither under normoxic or oxidative stress conditions (see FIG. 6 herein).

The newly discovered CXCR4/CCR3 heterodimeric complex is believed to have a modulating effect on CXCR4-mediated downstream intracellular signalling. In particular, the agonization of the CXCR4/CCR3 complex leads to a quick down-regulation of CXCR4 gene expression, and possibly the coupling of G-protein subunits to the CXCR4 receptor. In addition, cell types that express both chemokine receptors (CXCR4 and CCR3) are responsive to the NRP2945 and NNZ-4921-mediated ligand binding by activation of downstream gene expression of genes involved in survival, migration and final cellular differentiation.

In summary, the studies detailed herein show that NRP2945 and NNZ-4921 significantly reduce levels H₂O₂ induced cell death and oxygen glucose deprivation induced cell death in neurons. This neuroprotective activity depends on interactions with CXCR4 and CCR3. Therefore, NRP2945 and NNZ-4921 are acting as receptor agonists and are believed to recruit heterodimeric CXCR4/CCR3 complexes to the plasma membrane. Moreover, NRP2945 binding activation leads to an immediate down-regulation of CXCR4 gene expression. At the same time, NRP2945 displays anti-invasive and anti-migratory effects on cancerous cells expressing CXCR4/CCR3.

Thus, NRPs such as NRP2945 and NNZ-4921 can be used to modulate both the configuration and levels of CXCR4 in the cell. From this, NRPs and the receptors CXCR4/CCR3 have utility in a broad range of medical applications, including prevention and treatment of CNS disorders, heart failure and other cardiovascular conditions, diabetes, particularly type 1 diabetes where pancreatic beta cells co-express CXCR4 and CCR3, and various proliferative disorders, and particularly the prevention of cancer cell migration and metastasis. Moreover, NRPs have utility in a variety of assays, including methods of monitoring NRP-based treatments and methods of identifying new drug candidates.

### SUMMARY OF THE INVENTION

In one aspect, the invention encompasses a method of down-regulating CXCR4 expression in a cell, wherein the method comprises contacting the cell with exogenous NRP2945 (SEQ ID NO:1), NNZ-4921 (SEQ ID NO:2) and NRP2983 (SEQ ID NO:9), or a functional analogue thereof, thereby down-regulating CXCR4 expression.

In one other aspect, the invention encompasses a method of inhibiting migration of a cancer cell, the method comprising contacting the cancer cell with exogenous NRP2945 (SEQ ID NO:1), NNZ-4921 (SEQ ID NO:2), NRP2983 (SEQ ID NO:9), or a functional analogue thereof, thereby inhibiting the migration.

In yet one other aspect, the invention encompasses a method of inhibiting invasion of tissue by a cancer cell, the method comprising contacting the cancer cell with exogenous NRP2945 (SEQ ID NO:1), NNZ-4921 (SEQ ID NO:2), NRP2983 (SEQ ID NO:9) or a functional analogue thereof, thereby inhibiting the invasion.

In still one other aspect, the invention encompasses a method of inhibiting tumour metastasis, the method comprising contacting the tumour with exogenous NRP2945 (SEQ ID NO:1), NNZ-4921 (SEQ ID NO:2), NRP2983 (SEQ ID NO:9)or a functional analogue thereof, thereby inhibiting tumour metastasis.

In a specific aspect, the cancer cell in this method is a prostate-derived adenocarcinoma cell. In another specific aspect, the cancer cell is a prostate cancer cell.

In another aspect, the invention encompasses a method of treating or ameliorating cancer in a patient, the method comprising administering NRP2945 (SEQ ID NO:1), NNZ-4921 (SEQ ID NO:2), NRP2983 (SEQ ID NO:9) or a functional analogue thereof to the patient, thereby treating or ameliorating the cancer.

In yet another aspect, the invention encompasses a method of preventing or inhibiting tumour metastasis in a patient, the method comprising administering NRP2945 (SEQ ID NO:1), NNZ-4921 (SEQ ID NO:2), NRP2983 (SEQ ID NO:9) or a functional analogue thereof to the patient, thereby preventing or inhibiting tumour metastasis.

In still another aspect, the invention encompasses a method of inhibiting apoptosis in a neuron due to injury, the method comprising contacting the neuron with exogenous NRP2945 (SEQ ID NO:1), NNZ-4921 (SEQ ID NO:2), NRP2983 (SEQ ID NO:9) or a functional analogue thereof, thereby inhibiting apoptosis.

In a further aspect, the invention encompasses a method of preventing or inhibiting apoptosis of neurons due to CNS injury in a patient, the method comprising administering NRP2945 (SEQ ID NO:1), NNZ-4921 (SEQ ID NO:2), NRP2983 (SEQ ID NO:9) or a functional analogue thereof to the patient, thereby inhibiting apoptosis.

In yet a further aspect, the invention encompasses a method of promoting CXCR4/CCR3 heterodimer formation, wherein the method comprises contacting the cell with exogenous NRP2945 (SEQ ID NO:1), NNZ-4921 (SEQ ID NO:2), NRP2983 (SEQ ID NO:9) or a functional analogue thereof, thereby promoting CXCR4/CCR3 heterodimer formation.

In still a further aspect, the invention encompasses a method of activating a CXCR4 receptor in a cell, wherein the method comprises contacting the cell with exogenous NRP2945 (SEQ ID NO:1), NNZ-4921 (SEQ ID NO:2), NRP2983 (SEQ ID NO:9) or a functional analogue thereof, thereby activating the CXCR4 receptor.

In a further aspect the invention provides a neural regeneration peptide of SEQ ID NO:9.

In another aspect the invention further provides a composition comprising a neural regeneration peptide of SEQ ID NO: 9.

In another aspect there is provided a method of treating a neurological disorder characterized by loss of neural cells in an animal, comprising administering to said animal an amount of SEQ ID NO:9 or a composition as defined above. The neurological disorder is selected from amyotrophic lateral sclerosis, neurotoxin injury, oxidative injury, multiple sclerosis, peripheral neuropathy, hypoxia/ischemic, traumatic brain injury, optic nerve damage or diabetic peripheral neuropathy.

Additional aspects and embodiments of the invention will be apparent from the description that follows.

Definitions

In each instance herein, in descriptions, embodiments, and examples of the present invention, the terms “comprising”, “including”, etc., are to be read expansively, without limitation. Thus, unless the context clearly requires otherwise, throughout the description and the claims, the words “comprise”, “comprising”, and the like are to be construed in an inclusive sense as to opposed to an exclusive sense, that is to say in the sense of “including but not limited to”.

For convenience, general references to “NRP” or “NRPs” in this description will be taken to include peptides obtained from any source, e.g., isolated naturally occurring NRPs, recombinant NRPs, and synthetic NRPs, and to include NRPs having the naturally occurring peptide sequence as well as NRPs having modified peptide sequences. Of particular note are functional analogues of NRPs, i.e., analogues that retain one or more of the activities of the starting peptide sequence. Such activities are described further below.

“Exogenous” as it is used herein is intended to mean that the referenced molecule or the referenced activity is introduced into the host cell or host organism. An “exogenous” NRP refers to a peptide obtained by artificial, i.e., non-natural, means. This includes but is not limited to, synthetic chemistry, recombinant technology, purification protocols, etc. Included are peptides isolated from natural, recombinant, or synthetic sources. Also included are peptides produced by plasmids, vectors, or other expression constructs that may be introduced into a cell or cell-free translation system. An “exogenous” NRP is clearly distinguished from an endogenous, naturally occurring peptide that is made by the cell without human intervention.

It should be understood also that the terms “NRP”, “NRP compound”, “NRP analogue”, “SEQ ID NO:”, and other such terms, for simplicity, are used to identify the molecules described herein and not to provide their complete characterization. Thus, an “analogue of NRP” may be characterized herein as having a particular amino acid sequence, a particular 2-dimensional representation of the structure, but it is understood that the actual molecule claimed has other features, including 3-dimensional structure, mobility about certain bonds and other properties of the molecule as a whole. It is the molecules themselves and their properties as a whole that are encompassed by this disclosure.

In certain embodiments, the “analogues” of NRPs may have increased stability, due at least in part to decreased enzymatic degradation. The NRP analogues may have amino acid substitutions or modified amino acids. The NRP analogues are may have non-amino acid substituents replacing amino acids. The analogues of NRPs may include either amidated C-termini or can have C-terminal hydroxyl residues (OH). Other useful analogues are described in detail herein.

As used herein, “NRP”, “NRP compound,” “NRP analogue” and similar terms refer to functional sequences, e.g., sequences with one or more of the following activities: CXCR4 and/or CCR3 binding activity, CXCR4 activation activity, cell protection activity (e.g., neuroprotective activity), activity in preventing or inhibiting apoptosis (e.g., preventing apoptosis in neurons), activity in down-regulating CXCR4 expression, activity in promoting CXCR4/CCR3 heterodimer formation, and activity in inhibiting cancer cell migration, invasion, and/or metastasis.

Thus, the designation of a peptide as an “NRP” does not mean that it solely has neural effects. Rather, the term NRP is intended to include peptides having similar structural components as described herein, but may have effects on other cell types, tissues, and/or organs.

NRP Analogues

Analogues of NRPs may be produced to have a naturally occurring amino acid sequence and conformation. Alternatively, NRP analogues may include one or more of the following types of modifications: (1) stabilization of β-turns, (2) replacement of glycine residues, (3) replacement of the N-terminal glycine residue, and/or (4) cyclization.

Chou and Fasman β-turn prediction has revealed β-turns in various NRPs. These are found in amino acid domains APGR (SEQ ID NO:3) and RAGG (SEQ ID NO:4). For stabilization of β-turns, it is possible to introduce steric constraints such as alkylated amino acids. Readily available alkylated amino acids include alpha-aminoisobutyric acid (Aib), which can be used as a replacement for either or both of alanine and glycine residues in the NRPs.

One other useful modification is replacement of alanine with β-alanine. For example, it is possible to produce the peptide sequence Gly Arg Arg Ala Ala Pro Gly Arg β-Ala Gly Gly (SEQ ID NO:9).

For modification of the amino acid domain APGR (SEQ ID NO:3), the alanine or glycine can be replaced with alpha-aminoisobutyric acid (Aib). For modification of the amino acid domain RAGG (SEQ ID NO:4), the alanine can be replaced with aminoisobutyric acid (Aib).

It should be kept in mind that, for the NRPs, replacement of the internal glycine residue by an asparagine (N) can induce β-turns due to asparagine having higher β-turn propensity than glycine. Therefore, instead of replacement of the internal glycine residue with asparagine, it is possible to replace the glycine with asparagine at amino acid position 10, e.g., GRRAAPGRANG (SEQ ID NO:6).

Truncation of the G′ at the N terminus of the NRPs can result in loss of biological activity. In certain circumstances, G¹ may be replaced with an acetyl group. As to replacement of L-amino acids with D-amino acids, this may affect the secondary structure of the peptide. For NRPs, there may be circumstances where the third amino acid from the N-terminus, L-Arg, is replaced with D-Arg.

It may be desirable to produce a cyclic peptide mimetic of the NRPs. One method of cyclization involves adding a cysteine residue to each end of the sequence, and then oxidizing the resultant product to produce a cyclic disulphide. There may be situations where both the N and C terminal glycine residues are replaced with a cysteine residue and then oxidized. Direct cyclization of the C terminal residue to the N terminal residue can be accomplished by creating an amide bond.

The use of circular dichroism can indicate secondary structure and the use of computer simulation software for the modelling of small peptides can also be carried out using conventional methods. Both of these techniques can be used for determining structural features of the NRP analogues.

Synthesis of NRP Analogues

Starting materials and reagents for synthesis of peptides may be obtained from commercial suppliers such as Aldrich Chemical Company (Milwaukee, Wis.), Bachem (Torrance, Calif.), and Sigma (St. Louis, Mo.). Alternatively, reagents may be prepared by methods well known to the person of ordinary skill in the art.

Exemplary procedures are described in such references as Fieser and Fieser's Reagents for Organic Synthesis, vols. 1-17, John Wiley and Sons, New York, N.Y., 1991; Rodd's Chemistry of Carbon Compounds, vols. 1-5 and supplements, Elsevier Science Publishers, 1989; Organic Reactions, vols. 1-40, John Wiley and Sons, New York, N.Y., 1991; March J; Advanced Organic Chemistry, 4^{th} ed. John Wiley and Sons, New York, N.Y., 1992; and Larock: Comprehensive Organic Transformations, VCH Publishers, 1989.

In most instances, amino acids, their esters or amides, and protected amino acids, may be obtained from commercial suppliers. The preparation of modified amino acids and their amides or esters are also extensively described in the chemical and biochemical literature. Such procedures are considered to be well known to persons of ordinary skill in the art. For example, N-pyrrolidineacetic acid is described in Dega-Szafran Z. and Pryzbylak R., J. Mol. Struct., 436-7, 107-121, 1997; and N-piperidineacetic acid is described in Matsuda O, Ito S, and Sekiya M. Chem. Pharm. Bull.: 23(1), 219-221, 1975.

Synthetic production may be carried out using the solid-phase synthetic method described by Goodman M. (ed.), “Synthesis of Peptides and Peptidomimetics” in Methods of organic chemistry (Houben-Weyl) (Workbench Edition, 2004; Georg Thieme Verlag, Stuttgart, New York). This technique is well understood and is a common method for preparation of peptides.

The general concept of this method depends on attachment of the first amino acid of the chain to a solid polymer by a covalent bond. Succeeding protected amino acids are added, on at a time (stepwise strategy), or in blocks (segment strategy), until the desired sequence is assembled. Finally, the protected peptide is removed from the solid resin support and the protecting groups are cleaved off. By this procedure, reagents and by-products are removed by filtration, thus eliminating the necessity of purifying intermediaries.

Amino acids may be attached to any suitable polymer as a resin. Amide-polymer resins are particularly suitable for the present invention. The resin should contain a functional group to which the first protected amino acid can be firmly linked by a covalent bond. Various polymers are suitable for this purpose, such as cellulose, polyvinyl alcohol, polymethylmethacrylate, and polystyrene. Suitable resins are commercially available and well known to those of skill in the art.

Appropriate protective groups usable in such synthesis include tert-butyloxycarbonyl (BOC), benzyl (Bzl), t-amyloxycarbonyl (Aoc), tosyl (Tos), o-bromo-phenylmethoxycarbonyl (BrZ), 2,6-dichlorobenzyl (BzlCl₂), and phenylmethoxycarbonyl (Z or CBZ). Additional protective groups are identified in Goodman, cited above, as well as in McOmie JFW: Protective Groups in Organic Chemistry, Plenum Press, New York, 1973.

General procedures for preparing peptides involve initially attaching a carboxyl-terminal protected amino acid to the resin. After attachment, the resin is filtered, washed and the protecting group on the alpha-amino group of the carboxyl-terminal amino acid is removed. The removal of this protecting group must take place, of course, without breaking the bond between that amino acid and the resin. The next amino, and if necessary, side chain protected amino acid, is then coupled to the free amino group of the amino acid on the resin. This coupling takes place by the formation of an amide bond between the free carboxyl group of the second amino acid and the amino group of the first amino acid attached to the resin.

The above sequence of events is repeated with successive amino acids until all amino acids are attached to the resin. Finally, the protected peptide is cleaved from the resin and the protecting groups removed to reveal the desired peptide. The cleavage techniques used to separate the peptide from the resin and to remove the protecting groups depend upon the selection of resin and protecting groups and are known to those familiar with the art of peptide synthesis.

Peptides may be cyclized by the formation of a disulphide bond between two cysteine residues. Methods for the formation of such bonds are well known and include such methods as those described in G. A. Grant (Ed.) Synthetic Peptides: A User's Guide 2^{nd} Ed., Oxford University Press, 2002, W. C. Chan and P. D. White (Eds.) Fmoc Solid Phase Synthesis: A Practical Approach, Oxford University Press, 2000 and references therein.

Alternative techniques for peptide synthesis are described in Bodanszky et al, Peptide Synthesis, 2nd ed, John Wiley and Sons, New York, 1976, expressly incorporated herein fully by reference. For example, NRPs may also be synthesized using standard solution peptide synthesis methodologies, involving either stepwise or block coupling of amino acids or peptide fragments using chemical or enzymatic methods of amide bond formation. These solution synthesis methods are well known in the art. See, e.g. H. D. Jakubke in The Peptides, Analysis, Synthesis, Biology, Academic Press, New York, 1987, p. 103-165; J. D. Glass, ibid., pp. 167-184; and EP 0324659 A2, describing enzymatic peptide synthesis methods.

Commercial peptide synthesizers, such as the Applied Biosystems Model 430A, may also be used. While chemical synthesis of NRP analogues may represent the most convenient means for obtaining peptides, particularly the large scale production of peptides, it will be understood that other methods are also available to the skilled artisan, including recombinant peptide production, and isolation of endogenous peptides. Thus, the source of NRPs is not in any way limiting to the invention.

Assays using NRPs

As demonstrated herein, NRP2945 and NNZ-4921 act as CXCR4 agonists. It is believed that NRP2945 and NNZ-4921 actively recruit heterodimeric complexes of CXCR4/CCR3 to the plasma membrane. Moreover, NRP binding activation leads to an immediate down-regulation of CXCR4 gene expression. Based on this, NRPs such as NRP2945, NNZ-4921, and functional analogues thereof, can be used to modulate both the configuration and levels of CXCR4 in the cell. This has therapeutic utility, as well as utility in various assays that can be employed by the skilled artisan.

For assays to visualise NRPs, e.g., to assess binding or levels of NRPs, the peptides can be modified to include one or more labels that comprise a detectable substance. Suitable detectable substances include enzymes, prosthetic groups, fluorescent materials, luminescent materials, and radioactive materials. Examples of suitable enzymes include horseradish peroxidase, alkaline phosphatase, β-galactosidase, and acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material is luminol. Examples of suitable radioactive material include ¹⁴C, ¹²³I, ¹²⁴I, ¹²⁵I, ¹³¹I, ^{99m}Tc, ³⁵S, and ³H.

NRPs may be radioactively labelled with ¹⁴C, either by incorporation of ¹⁴C into the modifying group or one or more amino acid structures in the NRP. Labelled NRPs may be used to assess the in vivo pharmacokinetics of the compounds, as well as to assess appropriateness of a dosage amount or dosage regime, and predict whether dosage increases or decreases are necessary. Tissue distribution of CXCR4 receptors can be detected using a labelled NRP either in vivo or in an in vitro sample obtained from a subject.

For use in vivo, an NRP may be labelled with radioactive technetium or iodine. A modifying group can be chosen that provides a site at which a chelation group for the label can be introduced, such as the Aic derivative of cholic acid, which has a free amino group. Any of the various isotopes of radioactive iodine may be incorporated. ¹²³I (half-life =13.2 hours) may be used for whole body scintigraphy, ¹²⁴I(half-life =4 days) may be used for positron emission tomography (PET), ¹²⁵I (half-life=60 days) may be used for metabolic turnover studies and ¹³¹I (half-life=8 days) may be used for whole body counting and delayed low resolution imaging studies.

It is also possible to use click chemistry to add one or more detectable labels to NRPs. Click chemistry involves modular building blocks, for example, carbon-heteroatom bond formation. However, unlike naturally occurring modular reactions (e.g., peptide/protein synthesis), click chemistry reactions are irreversible. The reactions rely on highly energetic reagents or reactants (Kolb et al. Drug Discov Today. 8:1128, 2003). Examples of click chemistry reactions, include: cycloaddition reactions, such as the 1,3-dipolar family, and hetero Diels-Alder reactions (Karl Anker Angew Chem. 39:3558, 2000); nucleophilic ring-opening reactions (e.g., epoxides, aziridines, cyclic sulfates, etc.; Kolb et al. Angew Chem Int Ed. 40:2004, 2001); and carbonyl chemistry, such as the formation of oxime ethers, hydrazones, and aromatic heterocycles. Other reactions include carbon-carbon multiple bonds, such as epoxidation (Adolfsson et al. Tetrahedron Lett. 40:3991, 1999) and dihydroxylation (Kolb et al. Chem Rev. 94:2483, 1994) and azide-phosphine coupling (Staudinger ligation; Staudinger et al. Hely Chim Acta. 2:635, 1919; Gololobov et al. Tetrahedron. 37:437, 1981).

Click chemistry is particularly useful for the labeling of amino acid sequences. See, e.g., Wang et al., J Am Chem Soc. 125:3192, 2003; Link and Tirrell, J Am Chem Soc. 125:11164, 2003. Dieters et al. J Am Chem Soc. 125:11782, 2003. Peptides can be labeled with fluorescein by modifying either lysine or cysteine residues with azides or alkynes, followed by reaction with fluorescein-bearing complementary groups. Similarly, peptides can be synthesized with azide- or acetylene-based synthetic amino acids. In particular, an alkyne- or an azide-bearing peptide can be reacted with the counterpart unnatural amino acid. It is also possible to insert organic molecules to peptides in an azide-alkyne [3+2] cycloaddition reaction, by reacting an azide- or alkyne-bearing peptides with azide- or alkyne-bearing dyes. For the present invention, an NRP such as NRP2945 and NNZ-4921 may be modified by activating an internal aldehyde group, which becomes fluorescent upon CXCR4 and/or CCR3 receptor binding (Salic & Mitchison, PNAS 105(7): 2415-2420, 2008).

In other embodiments, NRPs are used in screens for potential drug candidates. Such screening methods can be carried out by providing a labelled NRP that has a detectable signal when bound to a CXCR4 receptor. The CXCR4 receptor is contacted with at least one test molecule at a known concentration to form a test sample. The test sample is then contacted with the NRP. Separately, the NRP is added to a sample not including any test molecule to form a control sample. The signal from the test sample is compared to the signal from the control sample.

The signal elicited by binding of the NRP and the receptor can be a fluorescent signal. As one example, the signal may be elicited when a second, accessory molecule is added, e.g., a fluorescent molecule may be bound to a molecule that binds the labelled NRP. As one particular example, the NRP may be labelled with biotin, and the accessory molecule may be a fluorescently labelled streptavidin molecule. For such experiments, the CXCR4 receptor may be expressed in a cell line. The process can be performed as a dose-response curve. The test compound may be incubated with the receptor at varying concentrations and the signal elicited after binding of the labelled NRP is measured and compared to control, as well as to each other.

As an alternative approach, test compounds may be assayed for receptor binding using a CXCR4 blocking monoclonal antibody (von Tscharner et al., Nature, 324, 369-372, 1986; see also US Patent No. 8138304). Competition of the test compound with an NRP can be carried out as described above. Other competition experiments may be performed using the CCR3 receptor in lieu of or in addition to the CXCR4 receptor.

Therapeutic uses of NRPs

CNS injuries and disorders

Based on the experimental results shown herein, NRPs such as NRP2945, NNZ-4921, and functional analogues thereof, can be used to prevent cell death via apoptosis in the central nervous system (CNS). In particular, NRPs may be produced and administered to patients affected by CNS injury or diseases.

Neuronal apoptosis is implicated in cell loss following acute CNS injury, e.g., ischemic or traumatic injury, as well as in chronic neurodegeneration. CNS injury can lead to apoptotic death in neurons, astrocytes, oligodendroglia, and inflammatory cells such as neutrophils, microglia, and macrophages. Neuronal death via apoptosis is also implicated in neurological disorders, including Alzheimer's, Parkinson's, and Huntington's diseases, stroke, progressive MS and amyotrophic lateral sclerosis (ALS). In such disorders, apoptosis involves oxidative stress, as well as perturbed calcium homeostasis resulting in mitochondrial and ER dysfunction.

The protective activity of NRPs can be utilized in prophylactic treatments, e.g., to block or reduce cell death in the CNS. Thus, NRPs can protect CNS cells from the effects of cerebrovascular disorders, including stroke, ischemic stroke, hypoxia/ischemia, ischemic infarction, atherosclerotic thrombosis, lacunes, embolism, hypertensive haemorrhage, ruptured aneurysms, vascular malformations, transient ischemic attacks, intracranial haemorrhage, spontaneous subarachnoid haemorrhage, hypoxic-ischemic encephalopathy, hypertensive encephalopathy, inflammatory diseases of the brain arteries, decreased perfusion caused by, for example, cardiac insufficiency (possibly resulting from coronary bypass surgery) and other forms of cerebrovascular disease.

NRPs can also be used to protect CNS cells from apoptosis following spinal cord or craniocerebral traumas, including basal skull fractures, cranial nerve injuries, diffuse axonal injury, asphyxia, perinatal hypoxic-ischemic injury, carotid-cavernous fistula, pneumocephalus, aerocele andrhinorrhea, cerebral contusion, traumatic brain injury, traumatic intracerebral haemorrhage, traumatic brain injury, penetrating traumatic brain injury and acute brain swelling in children.

NRPs can further be used to protect CNS cells from apoptosis resulting from demyelinating diseases that include neuromyelitis optica, acute disseminated encephalomyelitis, acute and subacute necrotizing haemorrhagic encephalitis, diffuse cerebral sclerosis of Schilder and multiple sclerosis in conjunction with peripheral neuropathy, as well as degenerative diseases of the nervous system including one or more of progressive dementia, diffuse cerebral atrophy, diffuse cortical atrophy of the non-Alzheimer type, Lewy body dementia, Pick's disease, fronto-temporal dementia, thalamic degeneration, deep ischaemic and haemorrhagic thalamic strokes, non-Huntingtonian types of chorea and dementia, cortico-spinal degeneration (Jakob), the dementia-Parkinson-amyotrophic lateral sclerosis complex (Guamanina and others) and amyotrophic lateral sclerosis (ALS).

Additionally, NRPs can be used to protect CNS cells from apoptosis resulting from peripheral neuropathies. There are more than 100 types of peripheral neuropathy, each with its own characteristic set of symptoms, pattern of development, and prognosis. Peripheral neuropathy may be either inherited or acquired. Inherited forms of peripheral neuropathy can be caused by genetic mutations or by significant genetic variations in epigenetically relevant genomic regions leading to potential gene expression disturbances.

Acquired peripheral neuropathy may result from, for example, physical injury (trauma) to a nerve, tumours, toxins (including chemotherapy), autoimmune responses, nutritional deficiencies, alcoholism, vascular and metabolic disorders (e.g. diabetic neuropathy). The HIV-associated peripheral neuropathy is a common side effect of drugs targeting the reverse transcriptase of the HIV virus. The symptoms of peripheral neuropathy can vary from temporary numbness, tingling, and pricking sensations, sensitivity to touch or muscle weakness, to more extreme symptoms such as burning pain, muscle wasting, paralysis, organ or gland dysfunction.

Moreover, NRPs can be used to protect CNS cells from apoptosis resulting from acquired metabolic disorders of the nervous system including metabolic diseases presenting as a syndrome comprising one or more of confusion, stupor or coma-ischemia-hypoxia, hypoglycaemia, hyperglycemia, hypercapnia, hepatic failure and Reye syndrome, metabolic diseases presenting as a progressive extrapyramidal syndrome, metabolic diseases presenting as cerebellar ataxia, hyperthermia, celiac-sprue disease, metabolic diseases causing psychosis or dementia including Cushing disease and steroid encephalopathy, thyroid psychosis and hypothyroidism and pancreatic encephalopathy. An example of a metabolic disorder that can result in neuropathy is excessive consumption of vitamin B6 (pyridoxine). This can be caused by amounts 100 times over the daily recommended intake when ingested for several weeks.

In further aspects, NRPs can be used to protect CNS cells from apoptosis resulting from diseases of the nervous system due to nutritional deficiency, drugs, alcohol, and alcoholism. Disorders of the nervous system due to drugs and other chemical agents include opiates and synthetic analgesics, sedative hypnotic drugs, stimulants, psychoactive drugs, bacterial toxins, plant poisons, venomous bites and stings, heavy metals, industrial toxins, anti-neoplastic and immunosuppressive agents, thalidomide, aminoglycoside antibiotics (ototoxicity) and penicillin derivatives (seizures), and cardioprotective agents (beta-blockers, digitalis derivatives and amiodarone).

The compositions and methods of the invention also find use in the prevention of cell death in the CNS due to acute brain injury, including but not limited to exposure to CNS toxins, and infections of the central nervous system, such as bacterial, fungal, spirochetal, parasitic, and sarcoid infections, including pyrogenic infections, bacterial meningitis, and leptomeningitis.

Patients suffering from one or more of the above diseases or injuries would benefit from a prophylactic treatment able to block or reduce apoptosis in the CNS.

Proliferative Conditions

As demonstrated herein, NRP2945 down-regulates expression of CXCR4, which has been implicated in various proliferative conditions, particularly hyperplasia, cancers, and metastases. NRP2945 is also shown herein to have anti-invasive and anti-migratory effects on cancerous cells expressing CXCR4/CCR3. Accordingly, NRPs can be used in preventive and/or therapeutic medicines for a range of proliferative conditions. NRPs, in particular NRP2945, NNZ-4921, and functional analogues thereof, can be used to inhibit cancer cell migration, invasion, and/or metastasis.

In different aspects, NRPs can be used as agents to prevent or treat proliferative conditions, such as hyperplasia and cancer, or to prevent or inhibit metastatic diseases. Accordingly, NRPs can be useful for the amelioration, prevention, and/or therapy of oral cancer, throat cancer, lip cancer, lingual cancer, gingival cancer, nasopharyngeal cancer, esophageal cancer, gastric cancer, small intestinal cancer, large intestinal cancer including colorectal cancer, liver cancer, gall bladder cancer, pancreatic cancer, nasal cancer, lung cancer, bone cancer, soft tissue cancer, skin cancer, melanoma, breast cancer, uterine cancer, ovarian cancer, prostate cancer, testicular cancer, penile cancer, bladder cancer, kidney cancer, brain cancer, in particular, glioblastoma multiforme and neuroblastoma, thyroid cancer, lymphoma, leukaemia, etc.

NRPs may be particularly suitable for treatment or amelioration of prostate cancer, and/or prevention or inhibition of metastasis of prostate cancer. Of note also are adenocarcinomas, particularly malignant adenocarcinomas. Exemplary adenocarcinomas include those of the prostate, as well as adenocarcinomas of the colon, rectum, lung, cervix, prostate, urachus, vagina, breast, esophagus, pancreas, stomach, and throat.

NRPs are considered to be especially useful in the prevention or treatment of malignant proliferative or neoplastic diseases, e.g. tumours, for example breast tumours; circulatory system tumours (e.g., heart, mediastinum, pleura, and other intrathoracic organ tumours, vascular tumours, and tumour-associated vascular tissue); excretory system tumours (e.g., kidney, renal pelvis, ureter, bladder, other and unspecified urinary organ tumours); gastrointestinal tract tumours (e.g., esophagus, stomach, small intestine, colon, colorectal, rectosigmoid junction, rectum, anus and anal canal tumours), tumours involving the liver and intrahepatic bile ducts, gall bladder, other and unspecified parts of biliary tract, pancreas, other and digestive organ tumours); head and neck; oral cavity tumours (e.g., lip, tongue, gum, floor of mouth, palate, and other parts of mouth, parotid gland, and other parts of the salivary glands, tonsil, oropharynx, nasopharynx, pyriform sinus, hypopharynx, and other sites in the lip, oral cavity and pharynx tumours).

Also included are reproductive system tumours (e.g., vulva, vagina, cervix uteri, corpus uteri, uterus, ovary, and other sites associated with female genital organs, placenta, penis, prostate, testis, and other sites associated with male genital organs); respiratory tract tumours (e.g., nasal cavity and middle ear, accessory sinuses, larynx, trachea, bronchus, and lung tumours, e.g., small cell lung cancer or non-small cell lung cancer); skeletal system tumours (e.g., bone and articular cartilage of limbs, bone articular cartilage and other sites); skin tumours (e.g., malignant melanoma of the skin, non-melanoma skin cancer, basal cell carcinoma of skin, squamous cell carcinoma of skin, mesothelioma, Kaposi's sarcoma); and brain and other central nervous system tumours (e.g., tumours of the meninges, brain, spinal cord, cranial nerves and other parts of central nervous system, e.g., glioblastomas or medulla blastomas); and head and/or neck cancer.

Further included are tumours involving other tissues including peripheral nerves and autonomic nervous system, connective and soft tissue, retroperitoneum and peritoneum, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites, tumours of blood and lymphatic system (e.g., Hodgkin's disease, Non-Hodgkin's lymphoma, Burkitt's lymphoma, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma, T-cell lymphoma or cutaneous T-cell lymphoma). Myeloid cancer includes, e.g., acute or chronic myeloid leukaemia.

Where a tumour, a tumour disease, a carcinoma, or a cancer is mentioned, this also includes metastasis in the original organ or tissue and/or in any other location, alternatively or in addition to the original site, whatever the location or locations of the tumour and/or metastasis. NRPs are indicated for treating tumour invasiveness or symptoms associated with such tumour growth, preventing metastatic spread of tumours or for preventing or inhibiting growth of micrometastasis in a subject in need thereof, especially for treating or preventing metastatic spread of tumours.

In one embodiment, NRPs are indicated for preventing or treating metastasis, tumour invasiveness, and/or tumour growth mediated by overexpression of CXCR4 and/or SDF-1 leading to a desensitization of the CXCR4 receptor system and subsequent malfunction. In a further embodiment, NRPs are indicated for inhibiting or controlling deregulated angiogenesis associated with tumours, e.g., angiogenesis mediated by CXCR4 and/or SDF-1, in a subject in need thereof.

The use of NRPs as anti-cancer agents can be made concomitantly with other anti-cancer drugs, for example, chemotherapeutic drugs, immunotherapeutic drugs, or drugs inhibiting the activity of cell growth factors and their receptors, amongst others. Thus, an NRP may exhibit a beneficial therapeutic activity when used in a single preparation form, but the activity can be further enhanced when used together with one or more concomitant drugs. For concomitant administration, exemplary chemotherapeutic drugs include alkylating drugs, antimetabolites, antibiotics and plant-derived anti-cancer drugs.

Included as alkylating drugs are nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, estramustine sodium phosphate, triethylenemelamine, carmustine, lomustine, streptozocin, pipobroman, etoglucide, altretamine, ambamustine, dibrospidium hydrochloride, fotemustin, prednimustin, pumitepa, ribomustin, temozolomide, treosulphan, trophosphamide, zinostatin stimalamer, carboquone, adzelecin, systemstin, bizelesin, platinum complex (carboplatin, cisplatin, miboplatin, nedaplatin, oxaliplatin, etc.).

Antimetabolites may include, for example, mercaptopurine, 6-mercaptopurine riboside, thioinosine, methotrexate, enocitabine, cytarabine, cytarabine ocfosfate, ancitabine hydrochloride, 5-FU agents (e.g., fluorouracil, tegafur, UFT, doxifluridine, carmofur, galocitabine, emitefur, etc.), aminopterin, calcium leucovorin, tabloid, butocin, calcium foliate, calcium levofolinate, cladribine, emitefur, fludarabine, gemcitabine, hydroxycarbamide, pentostatin, piritrexim, idoxuridine, mitoguzaon, thiazofurin, ambamustin and gemcitabine.

Anti-cancer antibiotics may include, for example, anthracycline anti-cancer agents (doxorubicine hydrochloride, daunorubicin hydrochloride, aclarubicin hydrochloride, pirarubicin hydrochloride, epirubicin hydrochloride, etc.), actinomycin D, actinomycin C, mitomycin C, chromomycin A3, bleomycin hydrochloride, bleomycin sulfate, phleomycin sulfate, neocarzinostatin, mithramycin, sarcomycin, carzinophilin, mitotane, zorbicin hydrochloride, mitoxantrone hydrochloride and idarubicin hydrochloride.

Plant-derived anti-cancer agents may include, for example, vinca alkaloid anti-cancer agents (vinblastine sulfate, vincristine sulfate, vindesin sulfate, vinorelbine, etc.), taxane anti-cancer agents (from taxus/yew plants, taxol-type drugs), (paclitaxel, docetaxel, etc.), etoposide, etoposide phosphate, teniposide, and vinorelbine.

Cell growth factors in the said drugs inhibiting the activity of cell growth factors and their receptors can include EGF (epidermal growth factor) or a material having substantially the same activity as EGF (e.g., EGF, HER2 ligand, etc.), insulin or a material having substantially the same activity as insulin (e.g., insulin, IGF (insulin-like growth factor)-1, IGF-2, etc.), FGF (fibroblast growth factor) a material having substantially the same activity as FGF (e.g., acidic FGF, basic FGF, KGF (keratinocyte growth factor), FGF-10, etc.), or other cell growth factors (e.g., G-CSF (granulocyte colony stimulating factor), EPO (erythropoietin), IL-2 (interleukin-2), NGF (nerve growth factor), PDGF (platelet-derived growth factor), TGF-β (transforming growth factors), HGF (hepatocyte growth factor), VEGF (vascular endothelial growth factor), etc.).

The receptors of cell growth factors can be any receptor that has binding capacity with the above-mentioned cell growth factors. Specifically, they include EGF receptor, HER2, insulin receptor, IGF receptor, FGF receptor-1 or FGF receptor-2, HGF receptor (c-met), VEG receptor, SCF receptor (c-kit), insulin receptor and sonic hedgehog (target for EPO). Drugs inhibiting the activity of cell growth factors may include Herceptin (HER2 anti-body), GLEEVEC (c-met, c-kit, abl inhibitor), Iressa (EGF receptor inhibitor) etc. Besides the above-mentioned drugs, topoisomerase I inhibitor (e.g., irinotecan, topotecan, etc.), topoisomerase II inhibitor (e.g., sobuzoxane, etc.), angiogenesis inhibitor, etc. can also be used.

Cardiovascular Conditions

CXCR4 is overexpressed in patients with heart failure, and, as demonstrated herein, NRP2945 down-regulates expression of CXCR4. Accordingly, NRPs such as NRP2945, NNZ-4921, and functional analogues thereof, can be used in preventive and/or therapeutic medicines for heart failure and other cardiovascular conditions.

Heart failure, often called congestive heart failure or congestive cardiac failure, occurs when the heart is unable to provide sufficient pump action to distribute blood flow to meet the needs of the body. Heart failure may be associated, for example, with myocardial infarction and various forms of ischemic heart disease, hypertension, valvular heart disease, and/or cardiomyopathy. See, e.g., McMurray and Pfeffer, Lancet 365 (9474): 1877-89, 2005.

Heart failure may also occur when the body's requirements for oxygen and nutrients are increased and the demand exceeds cardiac capacity. This can occur in association with severe anemia, Gram negative septicaemia, beriberi (vitamin B1/thiamine deficiency), thyrotoxicosis, Paget's disease, arteriovenous fistulae, or arteriovenous malformations.

During diagnosis, heart failure may be characterised as chronic, e.g., as associated with smoking, obesity, or diabetes, or acute. Acute decompensated heart failure is exacerbated or decompensated heart failure, referring to episodes in which a patient has symptoms that require urgent therapy or hospitalization. See, e.g., Jessup et al., Circulation, 119(14):1977-2016, 2009.

Heart failure may involve a condition on one side of the heart (i.e., left heart failure versus right heart failure), or conditions on both sides (i.e., mixed presentations). It may be associated with systolic dysfunction or diastolic dysfunction. The condition may be due primarily increased venous back pressure (preload), or failure to supply adequate arterial perfusion (afterload). The condition may be due to low cardiac output with high systemic vascular resistance or high cardiac output with low vascular resistance (i.e., low-output heart failure versus high-output heart failure). All forms and sources of heart failure are encompassed herein.

Other cardiovascular conditions which may be prevented or treated include coronary heart disease (also called ischaemic heart disease or coronary artery disease), cardiomyopathy (diseases of the cardiac muscle), hypertensive heart disease (diseases of the heart secondary to high blood pressure), cardiac dysrhythmias (abnormalities of heart rhythm), inflammatory heart disease, e.g., endocarditis (inflammation of the inner layer of the heart), inflammatory cardiomegaly, myocarditis (inflammation of the muscular part of the heart), valvular heart disease, cerebrovascular disease (disease of blood vessels that supplies to the brain such as stroke), peripheral arterial disease (disease of blood vessels that supplies to the arms and legs), congenital heart disease (heart malformations existing at birth), and rheumatic heart disease (heart damage due to rheumatic fever).

The use of NRPs as cardiovascular agents can be made concomitantly with other cardiovascular drugs. For example, one or more NRPs may be administered in conjunction with one or more angiotensin-converting enzyme (ACE) inhibitors, angiotensin II receptor blockers, digoxin, beta blockers, diuretics, or aldosterone antagonists.

Non-limiting exemplifications are provided as follows. ACE inhibitors include enalapril (e.g., Vasotec®), lisinopril (e.g., Prinivil®, Zestril®), and captopril (e.g., Capoten®). Angiotensin II receptor blockers include losartan (e.g., Cozaar®) and valsartan (e.g., Diovan®). Digoxin (e.g., Lanoxin®) is also referred to as digitalis. Beta blockers include carvedilol (e.g., Coreg®), metoprolol (e.g., Lopressor®), and bisoprolol (e.g., Zebeta®). Diuretics include bumetanide (e.g., Bumex®) and furosemide (e.g., Lasix®). Aldosterone antagonists include spironolactone (e.g., Aldactone®) and eplerenone (e.g., Inspra®).

Diabetic conditions

CXCR4 signalling is necessary for pancreatic islet cell genesis. As shown herein, NRPs such as NRP2945 and NNZ-4921 are CXCR4 agonists and bind to heterodimers of CXCR4/CCR3. Therefore, NRPs such as NRP2945, NNZ-4921, and functional analogues thereof, can be used in preventive and/or therapeutic medicines for diabetes, particularly type 1 diabetes where pancreatic beta cells co-express CXCR4 and CCR3.

Type 1 diabetes (also called diabetes mellitus type 1, formerly insulin dependent diabetes or juvenile diabetes) is a form of diabetes that results from autoimmune destruction of insulin-producing beta cells of the pancreas. The subsequent lack of insulin leads to increased blood and urine glucose. Type 1 diabetes is associated with dehydration, weight loss, diabetic ketoacidosis, and can ultimately lead to damage the nerves (diabetic neuropathy) and small blood vessels of the eyes (diabetic retinopathy), kidneys (diabetic nephropathy), and heart, and predispose a person to atherosclerosis of the large arteries that can cause heart attack and stroke. NRPs may be useful in halting or delaying the onset of these diabetic conditions.

NRPs may be used concomitantly with other drugs or treatments for diabetes. For example, one or more NRPs may be administered in conjunction with insulin treatments (e.g., subcutaneous insulin injection or insulin pump), or may be used in conjunction with pancreatic transplantation, pancreatic islet cell transplantation, or stem cell educator therapy. In certain aspects, NRPs may be used concomitantly with immunosuppressive drugs. Suitable drugs include, for example, cyclosporine A, anti-CD3 antibodies, including teplizumab and otelixizumab, anti-CD20 antibodies, including rituximab, anti-CD4 antibodies, and anti-CD8 antibodies.

Administration of NRPs

NRPs such as NRP2945, NNZ-4921, and functional analogues thereof, can be used via direct administration to the patient. In particular, one or more NRPs can be prepared and used as therapeutics. Peptides can be administered as part of a medicament or pharmaceutical preparation. This can involve combining an NRP with any pharmaceutically appropriate carrier, adjuvant, or excipient. Additionally an NRP can be used with other non-NRP neuroprotective agent or other therapeutic agent. The selection of the carrier, adjuvant, or excipient can depend upon the route of administration to be employed.

The administration route can vary widely to suit a particular condition. An NRP may be administered in different ways: intraperitoneally, intravenously, topically (e.g., eye drop) or intracerebroventricularly. Peripheral administration may be used to avoid direct interference with the central nervous system. Any known peripheral route of administration can be employed.

This includes parenteral administration, for example, injection into the peripheral circulation, subcutaneous administration, intraorbital administration, ophthalmic administration, intraspinal administration, intracisternal administration, topical administration, administration via infusion (using, e.g., slow release devices or minipumps such as osmotic pumps or skin patches), administration via implant, aerosol administration, administration via inhalation, scarification administration, intraperitoneal administration, intracapsular administration, intramuscular administration, intranasal administration, oral administration, buccal administration, pulmonary administration, rectal administration or vaginal administration.

The compositions can be formulated for parenteral administration to humans or other mammals in therapeutically effective amounts (e.g., amounts that provide prophylaxis) for CNS cell protection described above. Particular routes of administration include subcutaneous injection (e.g., dissolved in 0.9% sodium chloride) and oral administration (e.g., in a capsule).

It may be desirable to administer an NRP directly to the CNS of the patient. This can be carried out by any appropriate route of administration. Examples include administration by lateral cerebroventricular injection or through a surgically inserted shunt into the lateral cerebral ventricle of the brain of the patient, into the cerebrospinal fluid or directly into an affected portion of a patient's brain.

For proliferative disorders, an injection can be administered to the interior or proximal site of a tumour or directly to the lesion by intravenous, intramuscular, subcutaneous, intraorgan, intranasal, intradermal, intraocular (e.g., eye drops), intracerebral, intrarectal, intravaginal, or intraperitoneal administration.

For the various therapies noted above, one or more NRPs may be administered with one or more concomitant drugs to provide increased benefit to the patient (e.g., combination treatment of one or more NRPs with methylprednisone). The time of this administration is not limited. The NRP and the concomitant drug can be administered to the subject at the same time or at different times. The dose of a concomitant drug can follow the usual dose clinically adopted, and can be determined appropriately depending on the administration subject, administration route, disease conditions, combination, etc.

The administration mode of an NRP and a concomitant drug is not particularly limited, and it may be acceptable for the NRP or a salt thereof and a concomitant drug to be combined at the time of administration. Such administration mode may be, for example, the administration of a single preparation formulated by the simultaneous combination of an NRP and a concomitant drug. Alternatively, the simultaneous administration may be by the same administration route of two different drugs--one being a drug formulated using an NRP and the other being a concomitant drug.

As another embodiment, the administration by the same route may take place at different times with two different drugs--one being a drug formulated using an NRP and the other being a concomitant drug. As yet another embodiment, the simultaneous administration may be by different routes with two different drugs-one being a drug formulated using an NRP and the other being a concomitant drug. As still another embodiment, the administration may be by different routes at different times with two different drugs--one being a drug formulated using an NRP and the other being a concomitant drug (for example, the administration of an NRP followed by a concomitant drug, or vice versa), etc.

Any concomitant drug should have low toxicity. Accordingly such drugs can be safely administered orally or parenterally (e.g., by local, rectum, vein, etc.) in the form of pharmaceutical compositions prepared by mixing an NRP and/or the above-mentioned concomitant drug with a pharmacologically acceptable carrier in accordance with a method known in the art. Such pharmaceutical compositions include, without limitation, tablets (including sugar coated tablets and film-coated tablets), powders, granules, capsules (including soft capsules), solutions, injections, suppositories, sustained-release formulations, etc.

Therapeutic Doses of NRPs

The determination of an effective amount of an NRP, e.g., NRP2945, NNZ-4921, or functional analogues thereof, to be administered is within the skill of one of ordinary skill in the art, and will be routine to those persons skilled in the art. In certain embodiments, the amount of an NRP to be used can be estimated by in vitro studies using an assay system as described herein. The final amount of an NRP to be administered will be dependent upon the route of administration, upon the NRP used and the nature of the disorder or condition that is to be treated.

For inclusion in a medicament, an NRP can be directly synthesized by conventional methods such as those described herein. An NRP compound-containing composition may be administered by one or more routes, including those noted herein. By way of example, intravenous, intraperitoneal, intracerebral, intraventricular, inhalation, lavage, rectal, vaginal, transdermal, or subcutaneous administration can be used.

A suitable dose range may for example, be between about 0.1 μg to about 15 μg per 1 kg of body weight or in other embodiments, about 20 μg/kg to about 30 μg/kg body weight per day. Other dosages may range of from about 0.1 μg/kg body weight to about 100 μg/kg body weight. In other embodiments, a dose of 1 μg/kg body weight to about 10 μg/kg body weight can be useful. In further embodiments, a dose of an NRP can be in the range of about 0.1 μg/kg body weight to about 0.1 mg/kg. It will be appreciated that the noted doses are not intended to be limiting. Other doses outside the noted ranges can be determined by those with skill in the art.

Where an NRP is administered in combination with another drug, the content of the concomitant drug will vary depending on the drug preparation form that is used. It may be about 0.1 to 100% by weight in the whole preparation, or about 0.1 to 50% by weight, or about 0.5 to 20% by weight. The content of an additive such as a carrier in the concomitant drugs may also vary depending on the drug preparation form that is used. It may be about 1 to 99.9% by weight in the whole preparation, or about 10 to 90% by weight.

As a general proposition, the total pharmaceutically effective amount of an NRP administered parenterally per dose will be in a range that can be measured by a dose response curve. For example, an NRP in the blood can be measured in body fluids of the mammal to be treated to determine dosing. Alternatively, one can administer increasing amounts of an NRP compound to the patient and check the serum levels of the patient for the peptide. The amount of NRP to be employed can be calculated on a molar basis based on these serum levels of the NRP.

One method for determining appropriate dosing of the compound entails measuring NRP levels in a biological fluid such as a body or blood fluid. Measuring such levels can be done by any means, including RIA ELISA and a HPLC-based method, for example, using ¹³C-¹⁵N-labeled NRP2945 or NNZ-4921. After measuring NRP levels, the fluid is contacted with the compound using single or multiple doses. After this contacting step, the NRP levels are re-measured in the fluid. If the fluid NRP levels have fallen by an amount sufficient to produce the desired efficacy for which the molecule is to be administered, then the dose of the molecule can be adjusted to produce maximal efficacy.

This method can be carried out in vitro or in vivo. For example, after the fluid is extracted from a mammal and the NRP levels measured, the compound herein may be administered to the mammal using single or multiple doses (that is, the contacting step is achieved by administration to a mammal) and then the NRP levels are re-measured from fluid extracted from the mammal.

NRPs may be suitably administered by a sustained-release system. Examples of sustained-release compositions include semi-permeable polymer matrices in the form of shaped articles, for example, films, or microcapsules. Sustained-release matrices include polylactides (U.S. Pat. No. 3773919, EP 58481), poly(2-hydroxyethyl methacrylate) (Langer et al., 1981), ethylene vinyl acetate (Langer et al., supra), or poly-D-(−)-3-hydroxybutyric acid (EP 133988). Sustained-release compositions also include a liposomally associated compound.

Liposomes containing the compound are prepared by methods known to those of skill in the art, as exemplified by DE 3218121; Hwang et al., 1980; EP 52322; EP 36676; EP 88046; EP 143949; EP 142641; Japanese Pat. Appin. 83-118008, U.S. Pat. Nos. 4,485,045 and 4,544,545 and EP 102324. Liposomes may be of the small unilamellar type (from or about 200 to 800 Angstroms) in which the lipid content is greater than about 30 mol. percent cholesterol, the selected proportion being adjusted for the most efficacious therapy. PEGylated peptides, which have a longer lifespan than non-PEGylated peptides, can also be employed, based on, for example, the conjugate technology described in WO 95/32003.

In some embodiments, the NRP can be formulated generally by mixing each at a desired degree of purity, in a unit dosage injectable form (solution, suspension, or emulsion), with a pharmaceutically, or parenterally, acceptable carrier, i.e., one that is non-toxic to recipients at the dosages and concentrations employed and is compatible with other ingredients of the formulation. For example, the formulation preferably does not include oxidizing agents and other compounds that are known to be deleterious to peptides.

In some embodiments, formulations can be prepared by contacting a compound uniformly and intimately with liquid carriers, or finely divided solid carriers, or both. Then, if desired, the product can be shaped into the desired formulation. In some embodiments, the carrier is a parenteral carrier, alternatively, a solution that is isotonic with the blood of the recipient. Examples of such carrier vehicles include water, saline, Ringer's solution, a buffered solution, and dextrose solution. Non-aqueous vehicles such as fixed oils and ethyl oleate are also useful herein.

The carrier suitably contains minor amounts of additives such as substances that enhance isotonicity and chemical stability. Such materials are desirably non-toxic to recipients at the dosages and concentrations employed, and include, by way of example only, buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; glycine; amino acids such as glutamic acid, aspartic acid, histidine, or arginine.

Other additives include monosaccharides, disaccharides, and other carbohydrates such as cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts, e.g., NaCl, KCl, MgCl₂, CaCl₂, and the like. In certain embodiments, a peptide as described herein can be stabilized using 0.5 M sucrose or 0.5 M trehalose. Using such sugars can permit long-term storage of the peptides.

An NRP can be desirably formulated in such vehicles at a pH of from about 6.5 to about 8. Other pH levels may also be useful, for example, from about 4.5 to about 8. It will be understood that use of certain of the foregoing excipients, carriers, or stabilizers will result in the formation of salts of the compound. The final preparation may be a stable liquid or lyophilized solid.

In other embodiments, adjuvants can be used. Typical adjuvants which may be incorporated into tablets, capsules, and the like are a binder such as acacia, corn starch, or gelatin; an excipient such as microcrystalline cellulose; a disintegrating agent like corn starch or alginic acid; a lubricant such as magnesium stearate; a sweetening agent such as sucrose or lactose; a flavouring agent such as peppermint, wintergreen, or cherry. When the dosage form is a capsule, in addition to the above materials, it may also contain a liquid carrier such as a fatty oil. Other materials of various types may be used as coatings or as modifiers of the physical form of the dosage unit. A syrup or elixir may contain the active compound, a sweetener such as sucrose, preservatives like propyl paraben, a colouring agent, and a flavouring agent such as cherry.

Sterile compositions for injection can be formulated according to conventional pharmaceutical practice. For example, dissolution or suspension of the active compound in a vehicle such as water or naturally occurring vegetable oil like sesame, peanut, or cottonseed oil or a synthetic fatty vehicle like ethyl oleate or the like may be desired. Buffers, preservatives, antioxidants, and the like can be incorporated according to accepted pharmaceutical practice.

Desirably, an NRP composition to be used for therapeutic administration may be sterile. Sterility can be readily accomplished by filtration through sterile filtration membranes (e.g., membranes having pore size of about 0.2 micron). Therapeutic compositions generally can be placed into a container having a sterile access port, for example an intravenous solution bag or vial having a stopper pierceable by a hypodermic injection needle.

In other embodiments, an NRP can be stored in unit or multi-dose containers, for example, sealed ampules or vials, as an aqueous solution or as a lyophilized formulation for reconstitution. As an example of a lyophilized formulation, 10 ml vials are filled with 5 ml of sterile-filtered 0.01% (w/v) aqueous solution of compound, and the resulting mixture is lyophilized. The infusion solution can be prepared by reconstituting lyophilized compounds using bacteriostatic water or other suitable solvent.

In still further embodiments, a kit may contain a predetermined amount of lyophilized NRP compound, a physiologically compatible solution for preparation of a dosage form, a mixing vial, a mixing device, and instructions for use. Such kits can be manufactured and stored according to usual practices in the industry.

### EXAMPLES

The examples described herein are provided for the purpose of illustrating specific embodiments of the invention and are not intended to limit the invention in any way. Persons of ordinary skill can utilize the disclosures and teachings herein to produce other embodiments and variations without undue experimentation. All such embodiments and variations are considered to be part of this invention.

### Example 1

### Human Embryonic Stem Cell Culture

Experiments with human embryonic stem cells (hESC) were carried out in accordance with the guidelines and regulations of the NHMRC and with the approval of the Austin Health Human Research Ethics Committee (Approval number H2008/03194), and University of Melbourne Human Research Ethics Committee (Approval number 0605017). H9 (WA-09, WiCell) cell lines were grown as previously described (Dottori & Pera, 2007).

Briefly, hESC were cultured on mitomycin-C treated mouse embryonic fibroblasts (MEFs) in hESC medium consisting of high-glucose Dulbecco's Modified Eagle Medium (DMEM) without sodium pyruvate, supplemented with 1% insulin/transferrin/selenium, 0.1 mM β-mercaptoethanol, 1% nonessential amino acids (NEAA), 2 mM glutamine, 25 U/ml penicillin, 25 μg/ml streptomycin (all from Invitrogen) and 20% fetal calf serum (FCS) (Hyclone).

Alternatively, hESC were cultured on mitomycin-C treated human foreskin fibroblasts (HFF; ATCC, CRL-2097) in KSR media consisting of DMEM/nutrient mixture F-12, supplemented with 0.1 mM 3-mercaptoethanol, 1% nonessential amino acids (NEAA), 2 mM glutamine, 25 U/ml penicillin, 25 μg/ml streptomycin and 20% knockout serum replacement (all from Invitrogen).

All cells were cultured at 37° C. in 5% CO₂ with 100% humidity. Colonies were mechanically dissected every 7 days (subcultivated or passaged) and transferred to freshly prepared MEFs or HFFs. Media was changed every second day.

### Example 2

### Neuronal Differentiation and Growth

Neuronal differentiation was achieved using the noggin induction method described for mouse neurospheres as adapted by Dottori for human neurospheres (Dottori & Pera, 2007). The colonies were maintained at 37° C., with 5% CO₂ in hESC medium supplemented with 500 ng/ml of Noggin for 14 days while replacing Noggin every other day.

At this point, the cells were washed with PBS and the colonies were again mechanically dissociated, but this time the central (differentiated) part of the colony was also cut into smaller pieces using a 26-gauge needle. The pieces were transferred to individual wells in a low adherent 96-well plate containing Neurobasal®A (NBM) (Invitrogen) supplemented with 1× X B-27® (Invitrogen) and 1× N-2 (Invitrogen) 20 ng/ml human recombinant EGF and 20 ng/ml human recombinant bFGF (Pharmacia). Media was changed every 2 to 3 days to allow neurosphere formation over 2 weeks.

In order to facilitate neuronal differentiation, neurospheres were again separated into smaller pieces under a dissection microscope, and three to four pieces were transferred to each well of a 96-well plate. Prior to this transfer, the plate was pre-coated with poly-D-lysine (10 μg/ml in PBS), washed in PBS, recoated with mouse laminin (5 μg/ml in PBS), and washed again. The cells were then grown for 11 days (with media changed every two days) in NBM lacking growth factors prior to induction of injury and assessment of hypothermia.

### Example 3

### Injury and Hypothermia Induction

On the day of experiments, the medium was changed to NBM+N2 containing a B27 preparation lacking the usual antioxidants (Invitrogen; 10889-038) to eliminate their confounding effects (NBM-AO).

### Example 4

### Oxygen and Glucose Deprivation (ODG)

To maintain isoosmotic pressure conditions, 25 mM 2-deoxy-D-glucose was added to NBM-AO medium and equilibrated for 30 minutes at room temperature before the initial media change as described above. Increasing concentrations of NRP2945 ranging from 1 fM to 100 pM were added to the cells at the time of injury induction (GlycoSyn, Lower Hutt, New Zealand). Culture supernatant was removed after this 4 hour period and stored at 4° C. until analysed for lactate dehydrogenase (LDH) activity.

The media was replaced with fresh NBM-AO and the cells were incubated for a further 20 hours containing drug, before again measuring LDH. Oxidative stress was induced by adding 50 μM fresh H₂O₂ (Sigma, H1009) to the growth factor negative NBM on initiation of the experiment with continued culture for 4 hours when LDH was measured and NBM-AO without H₂O₂ was returned to the culture which was maintained for a further 20 hours before again measuring LDH. NRP2945 concentrations ranging from 1 fM to 100 pM were added to the cells at the time of oxidative stress induction.

To evaluate the effects of NRP2945 as time elapses after induction of injury, incubations with two most active concentrations of NRP2945 were started immediately, one, three and six hours after induction of injury and maintained until 24 hours in each of the models and outcome assessed at 4 hours and 24 hours as above. Measurement of lactate dehydrogenase activity (a marker of total cell death) and TUNEL staining (a marker of apoptotic death) was performed according to the kit manufacturer's instructions (Roche, 1164479001 and 11684795910 respectively).

### Example 5

### Preparation of Substrates for Cerebellar Microexplants

Glass coverslips were obtained from New Zealand BioLabs, size 18 mm×18 mm. The coverslips were placed in a 150 mm Petri dish, at 8 cover slips per dish. Both sides of the coverslips were soaked and washed in absolute ethanol. The ethanol was discarded, and autoclaved MilliQ® was added to rinse both sides of coverslips. The water was then discarded, and the coverslips were air dried under the laminar flow.

Poly-D-lysine (PDL) was obtained from Sigma (P7280, lyophilized powder, γ-irradiated, average mol wt 30,000-70,000, cell culture tested). This was diluted with autoclaved PBS to make a stock solution of 1 mg/ml. The stock solution was divided into aliquots of 500 μl/tube and stored at −20° C.

For experiments, the stock solution of PDL was diluted with PBS 1:10 to make working solution of with a final concentration of 100 μg/ml. Approximately 100 μl of the PDL working solution was added to the coverslips and this was incubated at 34° C. for 2 hours (minimum) to overnight.

Next, more than 10 ml autoclaved MilliQ® was added into each Petri dish and the PDL-coated coverslips were rinsed. The coverslips were then transferred to 6 well culture plates. After air drying, the plates were wrapped in foil and stored at 4° C. until use. Storage was possible for up to 2 weeks.

### Example 6

### Preparation of Cerebellar Microexplants for Culturing

Rat pups were used for this experiment (Wistar rats), either P3/4 or P7/8.

For extraction of P3/4 cerebellar cortices, 0.5 ml of ice-cold PBS/0.65% D(+)-glucose buffer was placed into a Petri dish and the tissue was placed into this solution. For extraction of P7/8 cerebellar cortices, 1.0 ml of ice-cold PBS/0.65% D(+)-glucose buffer was placed into a Petri dish and the tissue was placed into this solution.

The laminated cerebellar cortex was removed surgically and stored immediately in ice-cold PBS/0.65% D(+)-glucose buffer using the amounts as noted above. The cortex was then transferred to a Petri dish, and the meninges were removed from the cerebellar. The tissue was sliced by scissors, and passed once (P3/P4 pups) or twice (P7/P8 pups) through a 23-gauge sized needle attached to a 1 cc syringe to obtain uniformly sized microexplants. This was set aside in a Petri dish.

Next, the tissue was transferred into a 15 ml Falcon tube and centrifuged for 2 minutes at 350 rpm, at 4° C. The PBS/Glu(+) buffer was carefully discarded using a pipette. The pellet was resuspended in cold Neurobasal® medium (Invitrogen), at 1 ml per pup. This was centrifuged for 2 minutes at 350 rpm (60XG), at 4° C.

The medium was discarded and the pellet was resuspended in warm Neurobasal® medium, at 0.5 ml per P3/4 pup and 1.5 ml per P7/8 pup. This was then seeded on PDL-coated cover slips in 6 well plates at a volume of 40-45 μl per coverslip.

The coverslips were incubated at 34° C., in 5% CO₂, with 100% humidity for 45 minutes to 1.5 hours to allow adherence. Then, 1 ml of Neurobasal® medium was added per well and the microexplants were checked under a microscope for adherence.

### Example 7

### Administration of Toxins, AMD3100, eotaxin-3, and NNZ-4921

The toxins 3-nitropropionic acid and glutamate were prepared at 100× concentrations. For 50 mM 3-nitropropionic acid (Sigma), the stock solution was titrated with NaOH to a pH value of 7-7.2. For 50 mM L-glutamate, this was dissolved under heat (hot water).

As a negative control, 10 μl of 3-nitropropionic acid and 10 μl of glutamate was administered (4 wells). As a positive control, 20 μl of PBS was administered to normoxic microexplants without any toxins (4 wells).

AMD3100 (specific CXCR4 antagonist from Sigma) was administered simultaneously at 300 nM concentration while human recombinant eotaxin-3 (Pharmaco) was applied simultaneously at 10 and 100 nM final concentrations, respectively. NNZ-4921 (GRRAAPGRAGG; SEQ ID NO:2) was administered in a dilution series starting from 1 fM final concentration to 100 nM final concentration (4 wells for each dilution).

### Example 8

### Fixation and Counting of Cells within Cerebellar Microexplants

A 4% paraformaldehyde (PFA) solution was prepared with 4 g PFA/100 ml PBS and 100 μl l N NaOH (final 1 mM NaOH).

At the end of cell culture, the cell culture medium was removed by pipette and 1 ml PFA solution was added per well. Fixation was carried out for 10 minutes at room temperature or overnight at 4° C. After fixation, the PFA solution was removed and 1 ml PBS was added per well.

The complete growth area (attached neurons) was screened. The top 4 of the most densely populated areas per well were viewed under 20× magnification using a binocular microscope. All cells were counted that had migrated from the outer margin of the respective microexplants.

### Example 9

### Analysis of the Motility of Human DU-145 Prostate Cancer Cells

Human epithelial DU-145 cells were obtained from ATCC (HTB-81). The DU-145 cell line was originally isolated from a metastatic prostate-derived cancer formed in the brain. The cells are adherent, and co-express CXCR4 and CCR3 receptors similar to neuroblasts and neurons. For this experiment, DU-145 cells were cultivated in ATCC-formulated Eagle's Minimum Essential Medium with 10% FBS in 5% CO₂ at 37′C. The cells were then subcultivated as follows.

The media was removed and discarded. The cell layer was briefly rinsed with 0.25% (w/v) trypsin and 0.5 mM EDTA to remove all remaining trypsin inhibitors from the serum. To each flask, 2-3 ml of trypsin-EDTA solution was added. This was incubated at room temperature.

The cells were observed under an inverted microscope until dislodgement (between 10-15 minutes). Shaking was avoided to prevent the cells from clumping. For cells that were difficult to dislodge, the flask was placed briefly at 37° C. To the flask, 6-8 ml of complete growth medium was added, and the cells were aspirated gently by pipette. A subcultivation volume ratio of 1:4 to 1:6 was obtained.

### Example 10

### Preparation of Boyden Chamber for DU-145 Cell Assay

The Boyden chamber inserts (Corning) were coated for 2 hours at 37° C. with 100 μl /ml poly-D-lysine (PDL; culture grade from Invitrogen). Inserts were rinsed once with PBS. The bottom plate was coated overnight with 100 pg/ml of NRP2945 in 0.001% BSA/PBS. The bottom plate was then rinsed once with PBS. The coated Boyden chamber could then be refrigerated, stored, and ready for cell seeding.

### Example 11

### Seeding, Cultivation and Analysis of DU-145 Cells in Boyden Chamber Bioassay

Cells were trypsinized as described above. Trypsinization was stopped by addition of excess complete growth medium. Subsequently, cells were collected by centrifugation (1500 rpm for 5 minutes, at 4° C.), and reconstituted in new medium at a target concentration of about 5 million cells per ml. A typical experiment required about 0.3 ml of cell suspension per 12-well plate.

Cells were counted and 50,000 cells were seeded into one insert of a 12-well Boyden chamber. Approximately 50 μl of cell suspension was seeded into each well by pipetting. Cells were incubated at 37° C. in 5% CO₂. After 24 hours, inserts (8 μm pore size from Corning) were fixed in 4% paraformaldehyde/PBS. The upper layer of cells was removed by using a Q-tip®.

DU-145 cells attached to the lower insert membrane were visualized and counted with standard cell visualization methods using hematoxyclin staining. Cell counting was performed by counting all cells that have migrated to the lower part of the insert.

### Example 12

### Mechanism of Action Studies

To assess whether NRP2945 acts through activation of the CXCR4 receptor two additional experiments were performed.

In the first experiment, human neurons were exposed to oxygen glucose deprivation (OGD) injury as described above. Two concentrations of NRP2945 were used for this experiment in either presence or absence of 300 nM AMD3100, a known antagonist to CXCR4 receptor. The concentration of AMD3100 was chosen from previous study performed on rat hippocampal and cerebellar cells. The cells were grown in the same way as above described experiments and the analysis was done using the LDH assay.

The second experiment was done to assess the capability of NRP2945 for regulating the gene expression of its target receptor subunit CXCR4. For this purpose, W9-hESCs were either exposed to 10 minute, 30 minute or 60 minute of either 10 pM or 100 pM of NRP2945 under normoxic conditions; or in combination with 4 hours of OGD injury at concentrations of either 10 pM or 100 pM of NRP2945. At the end of the 4 hours, mRNA was extracted from all the cell samples, cDNA synthesis was performed using the SuperSript® cDNA kit (Invitrogen) followed by real time PCR. The efficacy of the real time PCR was 99% when using the following primers for the CXCR4 product propagation:

Forward:
(SEQ ID NO: 15)
AGCTGTTGGCTGAAAAGGTGGTCTATG (27-mer)

according to the publication: Nagase, Miyamasu et al. (2000) - J Immunol 164: 5935-5943

Reverse:
(SEQ ID NO: 16)
GCGCTTCTGGTGGCCCTTGGAGTGTG (26-mer)

### Example 13

### Statistical Analysis

To minimise the potential impact of systematic bias introduced because of differential evaporation from peripheral wells in multi-well plates these wells were filled with the same volume of medium as the internal test wells but not used for culture. Because of time and resource constraints, it was not possible to randomise the incubator usage for each respective temperature condition. Before counting of

Tunnel positive cells, wells were imaged and the images re-coded independently before quantitation. No additional blinding was performed for the machine read LDH assay process.

Within experiments, each comparison was performed at least in triplicate and the mean of these values taken forward into group comparisons. Two-way ANOVA was performed, followed by post-hoc Dunnett's multiple comparison test with significance set at p<0.05 using SPSS (Statistics 20). In this study, there were three between-group variables; treatment, position in the plate and experimental number, and one within-group variable; the time of assessment. All values are presented as mean±SEM.

### Example 14

### NRP13.q13.2 Gene Sequence

Human chromosome 13q13.2 is a known susceptibility locus for grand mal epilepsy, bipolar disorder and forms of autism. The only known EST in the NRP region is human uterus EST (GenBank: DB276481; containing introns). US Patent No. 7767786 reports a particular NRP 13q13.2 splice variant. Compared to the previously published splice variant in USB2, exon 1 is of a different identity. The total coding sequence is shortened to 3 exons. The complete coding sequence is 345 nucleic acids in length encoding a protein of 115 amino acids in length. The full length NRP sequence is believed to be secreted via the non-classical secretory pathway.

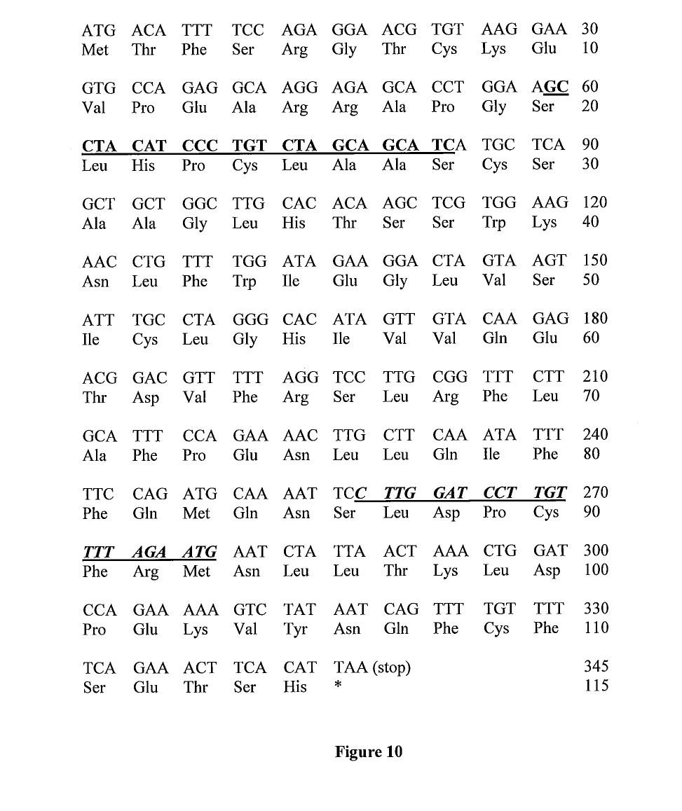

Human chromosome 13q13.2 NRP cds (3 exons) is depicted in FIG. 10. The full length nucleotide sequence corresponds to SEQ ID NO:12, while the full length amino acid sequence corresponds to SEQ ID NO:13. The amino acid sequence is shown also below. As depicted below, exon 1 is shown in bold/capitals. Exon 2 is shown in bold/italics. Exon 3 is shown in bold/lowercase. The underlined sequence (SEQ ID NO:14) is the shortest bioactive sequence for regenerative activities. MTFSRGTCKEVPEARRAPGSLHPclaascsaaglhtsswknlfwieglvsiclghivvqetdv frslrflafpenllqiffqmqnsldpefrmalltkldpekvynqfcfsetsh (SEQ ID NO:13)

### Example 15

### cDNA Synthesis and Semi-Quantitative RT-PCR

mRNA was extracted according to standard procedures (TRIzole® from Invitrogen or RNeasy Mini from Qiagen). The mRNA fraction was subjected to DNase I treatment using DNaseI incubation mixture. For this treatment, 10 μl DNaseI stock plus 70 μl buffer was added to mRNA preparation and incubated for 5 minutes at room temperature. cDNA synthesis was then carried out according to standard procedures (SuperScript® III Reverse Transcriptase from Invitrogen).

The optimized RT-PCR strategy used an intron-spanning forward primer with high GC-content and TM (78° C.) and a reverse primer with a lower TM (60° C.). These primers are shown in FIG. 10, and also further below. The mRNA was treated with DNase I before starting the synthesis of cDNA, as the intron-spanning primer only bridged an intron of 9 nucleic acids. It was postulated that this could cause formation of hairpin structures of the forward primer resulting in false positive genomic propagation of PCR products, hence the prior DNase I treatment.

The expected RT-PCR product was 221 by in length. PCR conditions included annealing at 58° C. and 36 total cycles. Samples were incubated at 94° C. for 5 minutes as the start of the GeneAmp® cycler operation. The 36 cycles included the sequence: 94° C. for 30 seconds; 58° C. for 30 seconds; 72° C. for 30 seconds; followed by cooling to 15° C. at the end of the PCR.

Forward primer:
humChr13NRP-F2AForward (25-mer)
(SEQ ID NO: 10)
5′-GCCTACATCCCTGTCTAGCAGCATC-3′
Reverse primer:
humChr13NRP-R2reverse (22-mer)
(SEQ ID NO: 11)
5′-CATTCTAAAACAAGGATCCAAG-3′

The PCR-reaction included: 10× buffer (2.50 μl), 50 mM MgCl₂ (0.75 μl), 10 mM dNTPs (0.50 μl), Primer 1 (0.50 μl), Primer 2 (0.50 μl), Taq Polymerase (0.10 μl), cDNA (1.00 μl ), H₂O (19.15 μl), to yield a total volume of 25.00 μl. Taq DNA Polymerase was sourced from Invitrogen (Platinum® Taq Polymerase).

### Example 16

### NTERA-2 Cell Line Cultivation, Differentiation, and Analysis

NTERA-2 (ATCC No. CRL-1973) is a human carcinoma derived pluripotent cell line that has only single copies of chromosome 1, 10, 11 and 13. This was the impetus for using 36 cycles of PCR for detection of NRP expression.

Undifferentiated NTERA-2 cells were obtained. Frozen stocks were prepared with a maximum of 2 passages. NRP expression could be assessed in cells having up to 4-5 passages. The cell culture medium included ATCC-formulated DMEM plus 10% FCS. The medium was exchanged every 2-3 days. For passaging, cells were dislodges by scraping and then transferred into 75cm² flasks. An initial seeding included 5 million cells in 12-15 ml of cell medium per flask.

Subcultivation was performed for gene expression tests. For subcultivation, cells were dislodged by scraping and then gathered by centrifugation at 1500 rpm for 7 minutes, at 4° C. The media was discarded and the cells were resuspended in 16 ml fresh DMEM/10%FBS. The expected yield was 12-15 million cells in the flask. Thus, the resuspended cells were at a concentration of about 1 million cells per ml. Adding 1 ml of cell suspension in each well of a 12-well tissue culture plate resulted in a plating density of approximately 1 million cells per well.

For passage 3, the remaining 4 ml of cell suspension plus 8 ml culture medium was transferred into new T75 cultivation flask.

### Example 17

### mRNA Harvest for Gene Expression Assay

Treatment conditions were as follows: 1) untreated control under normoxic conditions; 2) untreated control under oxidative stress (0.5 mM 3-nitropropionic acid (3-NP) from 50 mM stock (Sigma) titrated to pH: 6.8-7.0); 3) 1 pM NRP2945 under normoxic conditions; and 4) 1 pM NRP2945 under oxidative stress (0.5 mM 3-NP). All conditions were applied for 15 minutes, 30 minutes, and 60 minutes.

mRNA was then collected. Cells were washed once at the end of the treatment with subsequent cell scraping. Cells were passed through 25-gauge needle into a 15 ml falcon tube. Falcon tube cell content was aspirated, washed once with DMEM, followed by trypsinization. This was stopped by adding DMEM/10% FCS.

Centrifugation was carried out for 7 minutes at 1500 rpm, at 4° C. The pellets were then ready to be stored either at -80° C. or in liquid nitrogen until RNA extraction.

For RNA extraction, thawed cell pellets were prepared according to manufacturer's instructions (RNeasy Mini from Qiagen). RNA concentration was determined by NanoDropTM. This was followed by cDNA synthesis. For synthesis of sufficient quantities of cDNA, the RNA concentration in the cell samples was ensured to be between 10-20 ng/μl. This approximated a total yield of several micrograms per condition.

### Example 18

### Experimental Results

Neuronal injury and death

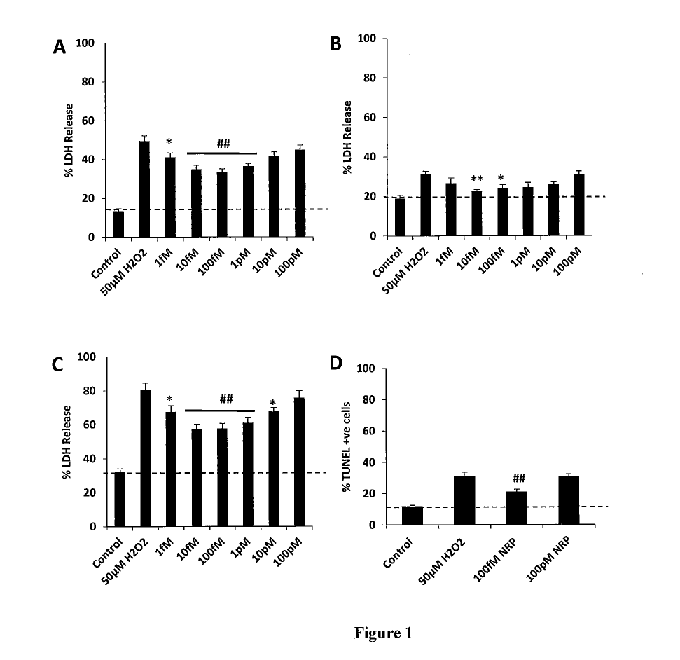

Both oxygen glucose deprivation and oxidative stress produced approximately 37% cell death at 4 hours (see FIG. 1). 50 μM H₂O₂ induced an increase in oxidative stress by 3.7 fold after 4 hours of injury (see FIG. 1A). Removal of H₂O₂ reduced the rate of cell death from 9%/hour within the first four hours to approximately 0.61%/hour for the following 20 hours (see FIG. 1).

Our data shows that NRP2945 provides dose dependant neuroprotection in human W9-hESCs in both injury models. Significant reduction of cell death was seen at a concentration of 1 fM where the cell death was reduced by 23.2% (p≦0.037 95% CI 1.04-45.3) (FIG. 1A). At a concentration of 10 fM, cell death was reduced by 40% (p≦0.0001 95% CI 17.9-62.2) (FIG. 1A). At 100 fM, cell death was reduced by 44% (p≦0.0001 95% CI 21.5-65.8) (FIG. 1A). At 1 pM, cell death was reduced by 35% (p0.0004 95% CI 13.50-057.7) (FIG. 1A). This takes into account correction for basal injury in the control.

Following the removal of H₂O₂ at four hours and completion of the experiment at 24 hours, NRP2945 continues to show neuroprotective effects. At concentrations of 10 fM and 100 fM, there was a reduction of LDH detected cell death by 70% (p≦0.01 95% CI 13.30-0126.4) and 57% (p≦0.048 95% CI 0.37-113.4) respectively (FIG. 1B).

The net effect at 24 hours was as follows: 1 fM reduced LDH release by 27% (p≦0.05 95% CI 0.03-53.7); 10 fM reduced LDH release by 48% (p≦0.0001 95% CI 20.8-74.5); 100 fM reduced LDH release by 47% (p≦0.0001 95% CI 20.1-74.0) 1 pM reduced LDH release by 26% (p≦0.001 95% CI 13.4-67.1) (FIG. 1C).

TUNEL staining for apoptotic cell death at 24 hours suggested that 19% of cell death occurred under normoxic conditions and that H₂O₂-mediated oxidative stress caused approximately 35% apoptotic cells. With administration of 100 fM NRP2945, 56.2% (p≦0.002 95% CI 17.7-86.2) of cells were spared from apoptosis under oxidative stress conditions (FIG. 1D).

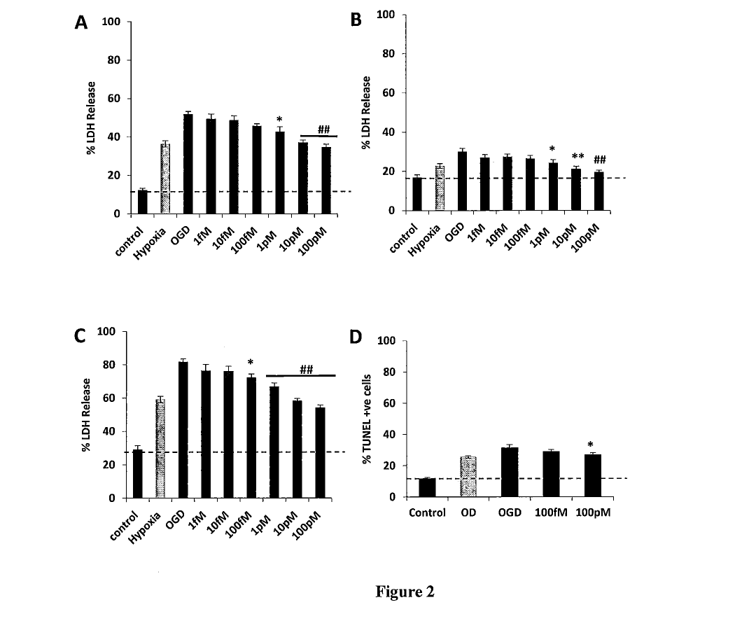

Oxygen deprivation alone increased LDH-detected cell death approximately 2.9-fold after 4 hours (see FIG. 2). Restoring the culture to a normal air/5% CO₂ incubator and replacing the culture media at 4 hours again slowed but did not completely halt the oxygen depletion induced cellular injury (6%/hour compared to 0.29%/hour, respectively; see FIG. 2). Combined oxygen and glucose deprivation caused much more significant injury, increasing LDH-detected cell death by 4.2-fold after 4 hours (see FIG. 2). This cell death continued at a slower rate on restoration of normal culture conditions (9.8%/hour and 0.65%/hour, respectively; see FIG. 2).

NRP2945 showed dose-dependent reduction of LDH detected cell death following OGD, however, much higher concentrations were needed than those necessary during H₂O_{2—} mediated injury. Significant reduction of LDH detected cell death was seen starting at a concentration of 1 pM, producing a reduction in cell death by 23% (p≦0.005 95% CI 5.3-40.7) (FIG. 2A). At 10 pM, cell death was reduced by 37% (p≦0.0001 95% CI 19.4-54.8) (FIG. 2A). At 100 pM, cell death was reduced by 43% (p≦0.0001 95% CI 25.9-61.3) (FIG. 2A).

At 10 pM and 100 pM, there was a dramatic decrease of 67% (p≦0.001 95% CI 24.3-110.0) and 79% (p≦0.0001 95% CI 36.0-121.8), respectively, for the delayed injury that occurs between removal of OGD at 4 hours and completion of the experiment at 24 hours (FIG. 2B).

The net effect at 24 hours was significant reduction of LDH release as follows: 100 fM produced a reduction of 17% (p≦0.032 95% CI 1.13-34.7); 1 pM produced a reduction of 28% (p≦0.00001 95% CI 11.5-45.1); 10 pM produced a reduction of 44% (p≦0.0001 95% CI 27.8-61.4); and 100 pM produced a reduction of 52% (p≦0.0001 95% CI 35.6-69.2) (FIG. 2C).

TUNEL staining for apoptotic cell death at 24 hours suggested that 20% of cell death occurred by this mechanism and that 100 pM protected 23% of W9-hESCs (p≦0.036 95% CI 1.2-45.0) from apoptotic cell death (FIG. 2D).

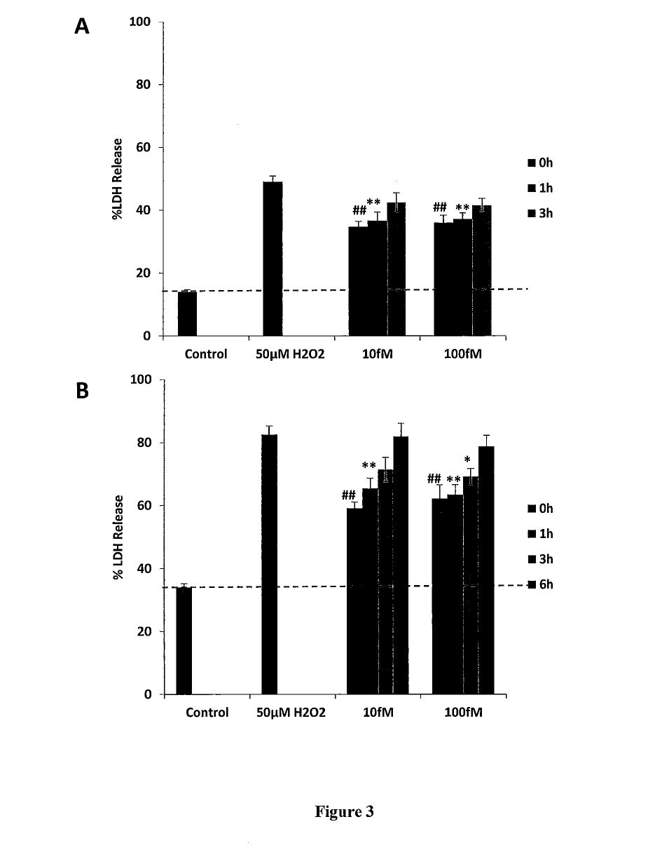

To confirm these observations, the H₂O₂ mediated injury and OGD experiments were repeated as before using concentrations of NRP2945 that showed highest neuroprotective effect (10 fM and 100 fM for H₂O₂ injury, and 10 pM and 100 pM for OGD). The period of injury exposure was 4 hours, with administration of NRP2945 delayed for 1 hour, 3 hours, and 6 hours (FIG. 3). Thus, the 6-hour time point was 2 hours after termination of the injury period.

As before, within the first 4 hours of H₂O₂ injury, both 10 fM and 100 fM NRP2945 provided a 40% (p≦0.0001 95% CI 16.2-65.3) and 37% (p≦0.001 95% CI 12.7-61.8) reduction in cell death. Both 10 fM and 100 fM NRP2945 significantly reduced cell death by 35% (p≦0.002 95% CI 10.8-59.9) and 33% (p≦0.003 95% CI 9.2-58.3) respectively, when administered one hour after H₂O₂ injury induction (exposed for three hours) (FIG. 3A). When NRP2945 was added 3 hours after H₂O₂ injury induction (1 hour exposure), there was no significant beneficial effect.

The net effect at 24 hours was a reduction of LDH release of 48% (p≦0.0001 95% CI 21.4-74.9), 35% (p≦0.004 95% CI 8.6-62.1), and 23% (p≦0.124 95% CI −3.8-49.6) with administration of 10 fM NRP2945, and 41% (p≦0.0001 95% CI 15.1-68.5), 39% (p≦0.001 95% CI 12.5-66.0) and 27% (p≦0.04 95% CI 0.82-54.3) with administration of 100 fM NRP2945, added at 0 hours, 1 hour, and 3 hours respectively, after H₂O₂ induction. (FIG. 3B). The net reduction in LDH release by addition of 100 fM NRP2945 at the 3 hour time point is statistically significant.

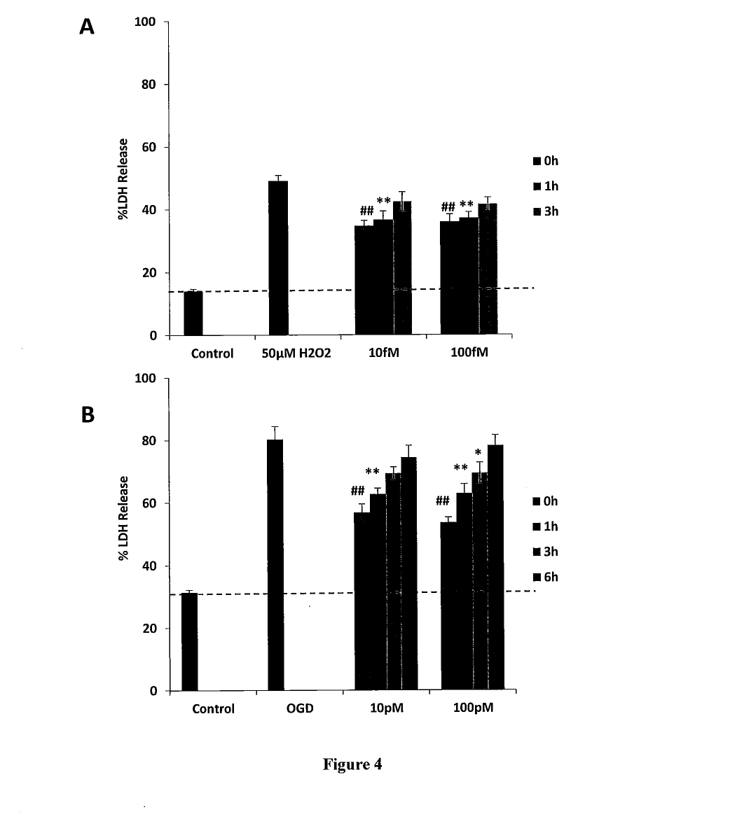

Similar decreases in LDH induce cell death were observed following OGD injury. Significant reduction in LDH detected cell death was seen when 10 pM and 100 pM concentrations of NRP2945 were administered during injury induction (41% reduction (p≦0.0001 95% CI 18.6-63.6) at 10 pM and 47% reduction (p≦0.0001 95% CI 25.2-70.2) at 100 pM) and one hour after injury induction (31% reduction (p≦0.003 95% CI 9.1-54.1) at 10 pM and 34% reduction (p≦0.001 95% CI 11.2-56.2) at 100 pM) (FIG. 4A).

The net effect at 24 hours following OGD injury was reduction of LDH release by 48% (p≦0.0001 95% CI 21.2-74.2), 36% (p≦0.004 95% CI 8.52-61.5), and 22% (p≦0.045 95% CI 0.95-53.5) with administration of 10 pM NRP2945, and reduction of LDH release by 54% (p≦0.0001 95% CI 14.9-67. 9), 35% (p≦0.001 95% CI 12.4-65.4) and 22% (p≦0.04 95% CI 0.8-53.7) with administration of 100 pM NRP2945, when added at 0 hours, 1 hour, and 3 hours, respectively (FIG. 4B). The net reduction in LDH release by addition of 10 pM and 100 pM NRP2945 at the 3 hour time point is statistically significant.

CXCR4 Receptor

To evaluate if NRP2945 exerts its cytoprotective effects via the CXCR4 receptor in human neuronal cell preparations, we exposed the cells to 300 nM of AMD3100, which is a known synthetic antagonist of the CXCR4 receptor. We used two different concentration of NRP2945. We chose concentrations of: a) 100 fM, which did not show neuroprotective effects during OGD, and b) 100 pM that did show neuroprotection during OGD injury.

The cells were exposed to OGD injury in the same way as the previous experiments. To ensure that 300 nM of AMD3100 has no toxic effects on the W9-hESCs,

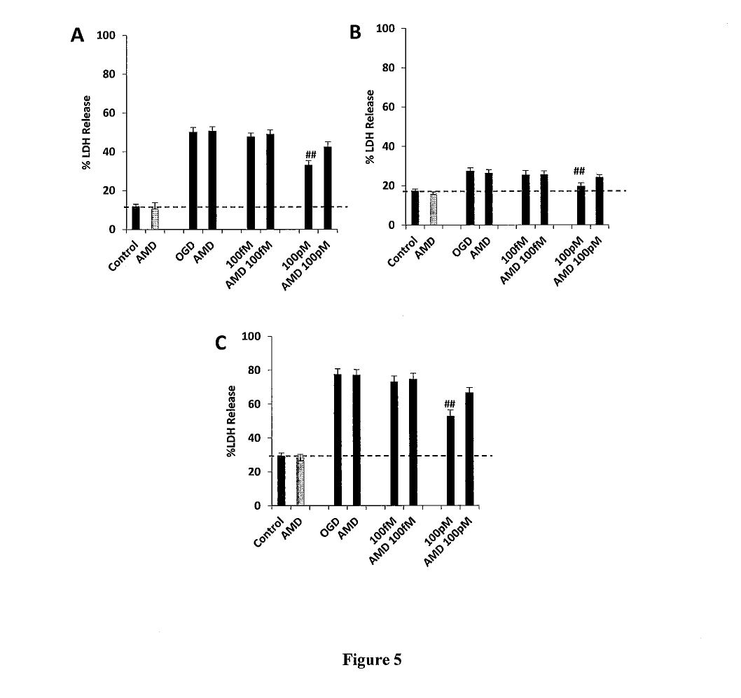

AMD3100 was tested under normoxic and OGD conditions. It did not show any toxicity under these test conditions (FIG. 5A).

Following 4 hours of OGD injury there was a significant decrse in LDH detected cells death seen with administration of 100 pM NRP2945 (44% reduction (p≦0.0001 95% CI 22.8-65.6)), but this decrease was abolished after the administration of the inhibitor (FIG. 5A).

Similar results were observed 24 hours after OGD induction. The net effect at 24 hours was significant reduction of LDH release (55% reduction (p≦0.0001 95% CI 26.1-76.5)) with administration of 100 pM NRP2945, but this reduction was blocked by AMD3100 (FIG. 5B). The results demonstrate that NRP2945 is indeed exerting its action at plasma membrane via the CXCR4 receptor.

CXCR4 and CCR3 receptors

Survival of cerebellar granule cells was then assessed as described in Example 7. Cerebellar granule cells representing the constituents of cerebellar microexplants were simultaneously challenged by glutamate and 3-NP toxicity while NNZ-4921, eotaxin-3 and AMD3100 were co-administered. Cell survival analysis was performed 48 hours later. Notably, eotaxin-3 alone had no toxic effect on normoxic cells or on cells maintained under oxidative/excitotoxic stress (FIG. 6).

Both concentrations of eotaxin-3 led to abrogation of the neuroprotective effect exerted by NNZ-4921 (FIG. 6). Additionally, the results indicated that the co-administration of eotaxin-3 and AMD3100 led to a synergistic effect as to the complete inhibition of the neuroprotective activity of NNZ-4921. As shown, eotaxin-3 in combination with AMD3100 completely blocked the activity of 10 fM or 100 fM NNZ-4921 in reducing cell death (FIG. 6). From this, it can be taken that CXCR4 forms a heterodimer with CCR3 that is critical for NRP-promoted neuron survival.

Notably, both antagonists (AMD3100 and eotaxin-3) of the respective CXCR4 and CCR3 receptors have the ability to completely block NNZ-4921 neuronal survival-promoting activity. There is no synergistic effect for the combined use of AMD3100 and eotaxin-3, which leads to the conclusion that the binding pocket for NRP molecules have to be constituted by both receptor subunits (CXCR4 and CCR3) to facilitate agonism of this heterodimeric complex.

CXCR4 and NRP Expression

The following set of experiments was performed to evaluate whether NRP2945 can be used to regulate CXCR4 gene expression after contact with the W9-hESCs. It is known that growth factors can regulate the gene expression status of its respective target chemokine receptors. There have several studies performed on stem cells and neural precursor cells evaluating SDF-1 and CXCR4 expression levels. Moreover, it has been shown that G-CSF when added to the cell culture medium of cultivated NPCs provokes up-regulation of CXCR4 within 24 hours of incubation (Kim et al., 2006). Nevertheless, there is no data showing regulation of CXCR4 gene expression within a short timeframe, i.e., minutes.

We have previously found that NRP2945 is able to up-regulate endogenous NRP gene expression (NRP gene located on 13q13.2) within human NTERA-2 cells in an autocrine fashion within the time frame of only 10 minutes of exposure (Sieg & Miyasmasu, unpublished results). Therefore, it was hypothesized that NRP2945 may also have an immediate effect on CXCR4 gene expression.

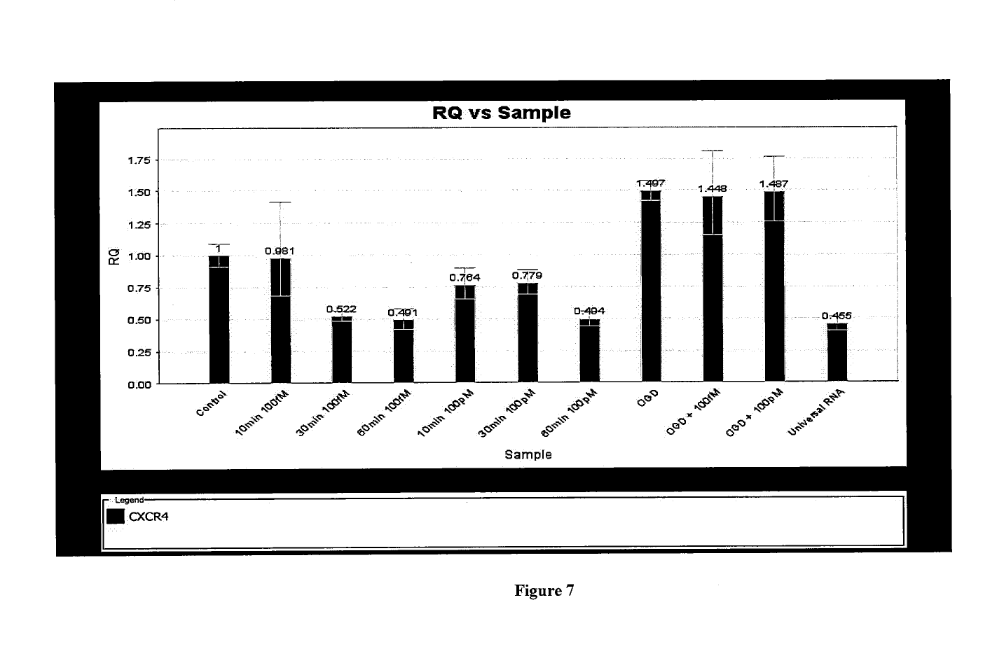

The data was presented as arbitrary expression units of the CXCR4 gene relative to normoxic conditions and normalised to β actin expression (housekeeping gene) (FIG. 7). Real time PCR revealed that expression of CXCR4 gene decreased significantly after only 10 minutes with increasing amounts of NRP2945 exposure under normoxic conditions. In the presence of OGD, CXCR4 gene expression increased by 50% and was unchanged by addition of NRP2945 (FIG. 7). A peak in

CXCR4 gene down-regulation was reached after 30 minutes and maintained for at least 1 hour (see FIG. 7). Thus, within 30 minutes, NRP2945 was able to down- regulate CXCR4 expression to constitutively expressed levels seen in a human tissue cDNA library.

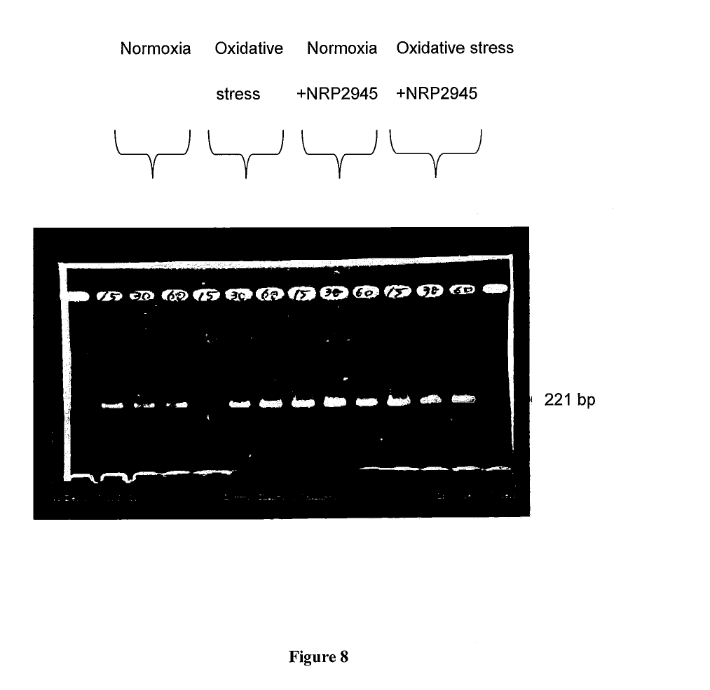

This expression pattern was compared to NRP expression on human chromosome 13. During normoxia, NRP was expressed at a low constitutive level (FIG. 8) because 36 PCR-cycles had to used to show respective NRP gene expression under normoxic conditions. After initiation of oxidative stress, NRP gene expression was completely blocked but recovered after 30 minutes (FIG. 8). Gene expression further increased at 60 minutes after initiation of oxidative stress. When NRP2945 was contacted with normoxic NTERA-2 cells, gene expression of NRP 13q13.2 was highly elevated after only 15 minutes. This peaked at 30 minutes after peptide contact (FIG. 8). During oxidative stress and simultaneous NRP2945 administration, NRP 13q13.2 gene expression is up-regulated after 15 minutes and stays elevated during the period of analysis (FIG. 8).

Cancer Cell Inhibition

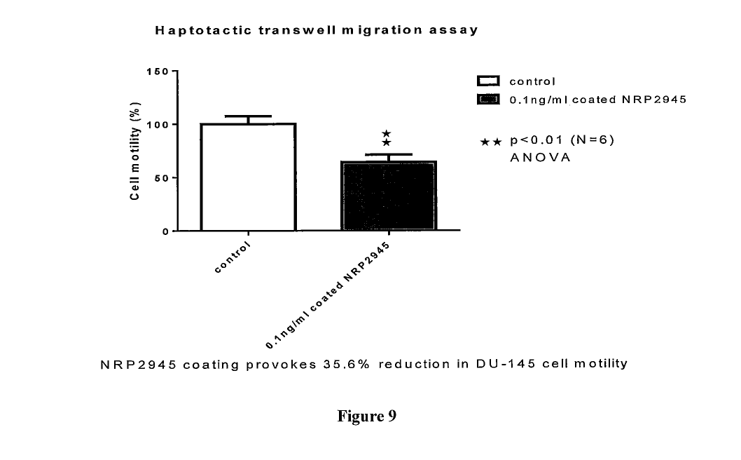

In additional studies, the motility of DU-145 cells (human prostate cancer cells) was assessed as described in Examples 9-11. We found that NRP2945 has the ability to decrease the motility/invasiveness of human prostate cancer derived DU-145 cells in a chemorepulsive manner. Adherence of 0.1 ng/ml of NRP2945 to the Boyden chamber bottom plate and subsequent DU-145 cell seeding led to a decrease of 36% of migrating cells after 24 hours of incubation (FIG. 9). Cancerous cells only expressing CXCR4 show no effect in respect to motility after NRP contact.

Thus, administration of 0.1 ng/ml NRP2945 led to a significant reduction in the motility of human DU-145 cells. This is in stark contrast to previous haptotactic migration assays using neural stem cells or primary neuronal cells and applying the same coating concentration of NRP2945. These previous experiments showed a significant increase in neuronal migration. See, e.g., US Patent No. 7563862. Notably, the same concentration of NRP2945 that provokes chemoattraction of neuronal cells leads to an inhibitory migratory activity on cancer cells co-expressing the CXCR4 and CCR3 receptor as shown for human DU-145 cells.

### Example 19

### Conclusions

From the experimental data, we conclude the following. NRP binding activation (e.g., via NRP2945 or NNZ-4921) causes formation of CXCR4/CCR3 heterodimers, and leads to an immediate down-regulation of CXCR4 gene expression. Notably, CXCR4 down-regulation is a preferred bioactivity because final cellular differentiation is initiated after NRP contact with pre-differentiated neural stem cells. Therefore, NRPs such as NRP2945 and NNZ-4921 are acting as receptor agonists that are believed capable of recruiting heterodimeric CXCR4/CCR3 complexes to the plasma membrane. At the same time, NRPs, including NRP2945 and NNZ-4921, are useful for their anti-invasive and anti-migratory effects on cancerous cells expressing CXCR4/CCR3.

Although the invention has been described by way of example, it should be appreciated that variations and modifications may be made without departing from the scope of the invention. Furthermore, where known equivalents exist to specific features, such equivalents are incorporated as if specifically referred to in this specification.

### REFERENCES

Gorba, T et al. 2006. Neural Regeneration Proteins are novel chemoattractive and survival promoting peptides. Exp Cell Research, 312: 3060-3074

Sieg, F and Antonic, A 2007. Alternative Strategies in Neuroregeneration and Neurogenesis. Research signpost edition (chapter, pp 27-58). Editors: Valeria Sogos and Andrea Diana.

Speidel, D et al. 2003. A family of Ca2+-dependent activator proteins for secretion: comparative analysis of structure, expression, localization, and function. J Biol Chem 278(52): 52802-52809.

Nedellec, R et al. 2009. Virus entry via the alternative coreceptors CCR3 and FPRL1 differs by human immunodeficiency virus type 1 subtype. J. Virol 83(17): 8353- 8363.

Sehgal A et al. 1998. Molecular characterization of CXCR-4: a potential brain tumor-associated gene. J Surg Oncol 69 (4): 239-248.

Oh, S B et al. 2001. Chemokines and glycoprotein120 produce pain hypersensitivity by directly exciting primary nociceptive neurons. J Neurosci 21(14): 5027-5035.

Singh, A T et al. 2010. Regulation of trophoblast migration and survival by a novel neural regeneration peptide (NRP). Reprod Biomed Online 21(2): 237-244.

Gupta, S K et al. 1999. Modulation of CXCR4 expression and SDF-lalpha functional activity during differentiation of human monocytes and macrophages. J Leuco Biol 66(1): 135-143.

Yang, J et al. 2012. Quantitative changes in gene transcription during induction of differentiation in porcine neural progenitor cells. Mol Vis 18: 1484-1504.

Yoshida, N et al. 2011. CXCR4 expression on activated B cells is downregulated by CD63 and IL-21. J Immunol 186(5); 2800-2808.

Darash-Yahana, M et al. 2004. Role of high expression levels of CXCR4 in tumor growth, vascularization, and metastasis. FASEB J 18(11): 1240-1242.

Van der Meer, P et al. 2001. Expression pattern of CXCR3, CXCR4, and CCR3 chemokine receptors in the developing human brain. J Neuropathol Exp Neurol 60(1): 25-32.

Stumm, R K et al. 2002. A dual role for the SDF-1/CXCR4 chemokine receptor system in adult brain: isoform-selective regulation of SDF-1 expression modulates

CXCR4-dependent neuronal plasticity and cerebral leukocyte recruitment after focal ischemia. J Neurosci 22(14): 5865-5878.

Mines, M A et al. 2009. Deubiquitination of CXCR4 by USP14 is critical for both CXCL12-induced CXCR4 degradation and chemotaxis but not ERK activation. J Biol Chem 284(9): 5742-5752.

Contento, R L et al. 2008. CXCR4-CCR5: a couple modulating T cell functions. PNAS 105(29): 10101-10106.

Pearson L L et al. 2001. CD40-mediated signalling in monocytic cells: up-regulation of tumor necrosis factor receptor-associated factor mRNAs and activation of mitogen-activated protein kinase signalling pathways. Int Immunol 13(3): 273-283.

Luo, Y et al. 2006. SDFlalpha/CXCR4 signalling stimulates beta-catenin transcriptional activity in rat neural progenitors. Neurosci Lett 398(3): 291-295.

Xu, H and Heilshorn, S C 2012. Microfluidic Investigation of BDNF-Enhanced Neural Stem Cell Chemotaxis in CXCL12 Gradients. Small 9(4): 585-595.

Ratajczak, M Z and Kim, C 2012. The use of chemokine receptor agonists in stem cell mobilization. Expert Op Biol Ther 12(3); 287-297.

Dottori, M and Pera, M 2008. Neural Stem Cells. Methods in Molecular Biology—SpringerLink. Volume 438. Book chapter: Neural differentiation of human embryonic stem cells (pp: 19-30). Editor: Leslie P. Weiner.

Ponath et al. Molecular cloning and characterization of a human eotaxin receptor expressed selectively on eosinophils. J. Exp. Med. 183: 2437-48, 1996.

Aukrust et al. Elevated circulating levels of C-C chemokines in patients with congestive heart failure. Circulation. 1998;97(12):1136-43.

Damas et al. Myocardial expression of CC- and CXC-chemokines and their receptors in human end-stage heart failure. Cardiovasc Res. 2000;47(4):778-87.

Damas et al. Enhanced gene expression of chemokines and their corresponding receptors in mononuclear blood cells in chronic heart failure-modulatory effect of intravenous immunoglobulin. J Am Coll Cardiol. 2001;38(1):187-93.

Kayali et al. 2012. The SDF-la/CXCR4 Axis is Required for Proliferation and Maturation of Human Fetal Pancreatic Endocrine Progenitor Cells. PLoS ONE 7(6): e38721.

Daugherty et al. Cloning, expression, and characterization of the human eosinophil eotaxin receptor. J. Exp. Med. 183 (5): 2349-54, 1996.

Youn et al. Molecular cloning of leukotactin-1: a novel human beta-chemokine, a chemoattractant for neutrophils, monocytes, and lymphocytes, and a potent agonist at CC chemokine receptors 1 and 3. J. Immun. 159: 5201-5205, 1997.

Kitaura et al. Molecular cloning of human eotaxin, an eosinophil-selective CC chemokine, and identification of a specific eosinophil eotaxin receptor, CC chemokine receptor 3. J. Biol. Chem. 271:7725-7730, 1996.

Kitaura et al. Molecular cloning of a novel human CC chemokine (Eotaxin-3) that is a functional ligand of CC chemokine receptor 3. J. Biol. Chem. 274:27975-27980, 1999.

Pan et al. A novel chemokine ligand for CCR10 and CCR3 expressed by epithelial cells in mucosal tissues, J. Immunol. 165: 2943-2949, 2000.

White et al. Cloning and functional characterization of a novel human CC chemokine that binds to the CCR3 receptor and activates human eosinophils. J. Leukoc. Biol. 62:667-675, 1997.

Each publication, including all published books, articles, patents, and patent applications noted in this specification, is expressly and fully incorporated herein by reference.

1. A method of down-regulating CXCR4 expression in a cell, wherein the method comprises contacting the cell with exogenous NRP2945 (SEQ ID NO:1), NNZ-4921 (SEQ ID NO:2), NRP 2983 (SEQ ID NO:9) or a functional analogue thereof, thereby down-regulating CXCR4 expression.

2. The method of claim 1, wherein the cell is a cancer cell.

3. The method of claim 1, wherein the cell is an adenocarcinoma-type cancer cell.

4. The method of claim 1, wherein the cell is a prostate cancer cell.

5. The method of claim 1. wherein the cell is a neuronal cell, neuronal stem cell or neuronal precursor cell.

6. A method of inhibiting migration of a cancer cell, the method comprising contacting the cancer cell with exogenous NRP2945 (SEQ ID NO:1), NNZ-4921 (SEQ ID NO:2), NRP 2983 (SEQ ID NO:9) or a functional analogue thereof, thereby inhibiting the migration.

7. The method of claim 6, wherein the cancer cell is an adenocarcinoma cell.

8. The method of claim 6, wherein the cancer cell is a prostate cancer cell.

9. A method of inhibiting invasion of tissue by a cancer cell, the method comprising contacting the cancer cell with exogenous NRP2945 (SEQ ID NO:1), NNZ-4921 (SEQ ID NO:2), NRP 2983 (SEQ ID NO:9) or a functional analogue thereof, thereby inhibiting the invasion.

10. The method of claim 9, wherein the cancer cell is an adenocarcinoma cell.

11. The method of claim 9, wherein the cancer cell is a prostate cancer cell.

12. A method of inhibiting tumour metastasis, the method comprising contacting the tumour with exogenous NRP2945 (SEQ ID NO:1), NNZ-4921 (SEQ ID NO:2), NRP 2983 (SEQ ID NO:9) or a functional analogue thereof, thereby inhibiting tumour metastasis.

13. The method of claim 12, wherein the tumour is an adenocarcinoma-type tumour.

14. The method of claim 12, wherein the tumour is a prostate tumour.

15. A method of treating or ameliorating cancer in a patient, the method comprising administering NRP2945 (SEQ ID NO:1), NNZ-4921 (SEQ ID NO:2), NRP 2983 (SEQ ID NO:9) or a functional analogue thereof to the patient, thereby treating or ameliorating the cancer.

16. The method of claim 15, wherein the cancer is an adenocarcinoma-type cancer.

17. The method of claim 15, wherein the cancer is prostate cancer.

18. A method of preventing or inhibiting tumour metastasis in a patient, the method comprising administering NRP2945 (SEQ ID NO:1), NNZ-4921 (SEQ ID NO:2), NRP 2983 (SEQ ID NO:9) or a functional analogue thereof to the patient, thereby preventing or inhibiting tumour metastasis.

19. The method of claim 18, wherein the tumour is an adenocarcinoma-type tumour.

20. The method of claim 18, wherein the tumour is a prostate tumour.

21. A method of inhibiting apoptosis in a neuron due to injury, the method comprising contacting the neuron with exogenous NRP2945 (SEQ ID NO:1), NNZ-4921 (SEQ ID NO:2), NRP 2983 (SEQ ID NO:9) or a functional analogue thereof, thereby inhibiting apoptosis.

22. The method of claim 21, wherein the injury is mechanical injury, oxidative injury, injury due to oxygen and glucose deprivation, or injury due to a toxin.

23. A method of preventing or inhibiting apoptosis of neurons due to CNS injury in a patient, the method comprising administering NRP2945 (SEQ ID NO:1), NNZ- 4921 (SEQ ID NO:2), NRP 2983 (SEQ ID NO:9) or a functional analogue thereof to the patient, thereby inhibiting apoptosis.

24. The method of clam 23, wherein the CNS injury is ischemic injury, injury due to trauma, or injury due to a neurological disease.

25. A method of promoting CXCR4/CCR3 heterodimer formation, wherein the method comprises contacting the cell with exogenous NRP2945 (SEQ ID NO:1), NNZ-4921 (SEQ ID NO:2), NRP 2983 (SEQ ID NO:9) or a functional analogue thereof, thereby promoting CXCR4/CCR3 heterodimer formation.

26. The method of claim 25, wherein the cell is a cancer cell.

27. The method of claim 25, wherein the cell is an adenocarcinoma-type cancer cell.

28. A method of activating a CXCR4 receptor in a cell, wherein the method comprises contacting the cell with exogenous NRP2945 (SEQ ID NO:1), NNZ-4921 (SEQ ID NO:2), NRP 2983 (SEQ ID NO:9) or a functional analogue thereof, thereby activating the CXCR4 receptor.

29. The method of claim 28, wherein the cell is a CNS cell.

30. The method of claim 28, wherein the cell is a neuron.

31. A neural regeneration peptide of SEQ ID NO:9.

32. A composition comprising a neural regeneration peptide of SEQ ID NO: 9.

33. A method of treating a neurological disorder characterized by loss of neural cells in an animal, comprising administering to said animal an amount of SEQ ID NO:9 or a composition as claimed in claim 32.

34. The method of claim 33 wherein said neurological disorder is amyotrophic lateral sclerosis, neurotoxin injury, oxidative injury, multiple sclerosis, peripheral neuropathy, hypoxia/ischemia, traumatic brain injury, optic nerve damage or diabetic peripheral neuropathy.


SEQUENCE LISTING (16 sequences)

SEQ ID NO: 1 - protein, 11 aa, Artificial Sequence
  Peptide
GRRAAPGRXGG

SEQ ID NO: 2 - protein, 11 aa, Artificial Sequence
  Peptide
GRRAAPGRAGG

SEQ ID NO: 3 - protein, 4 aa, Artificial Sequence
  Peptide
APGR

SEQ ID NO: 4 - protein, 4 aa, Artificial Sequence
  Peptide
RAGG

SEQ ID NO: 5 - protein, 11 aa, Artificial Sequence
  Peptide
GRRAXPGRAGG

SEQ ID NO: 6 - protein, 11 aa, Artificial Sequence
  Peptide
GRRAAPGRANG

SEQ ID NO: 7 - protein, 12 aa, Artificial Sequence
  Peptide
GRDRAAPGRAGG

SEQ ID NO: 8 - protein, 13 aa, Artificial Sequence
  Peptide
REGRRDAPGRAGG

SEQ ID NO: 9 - protein, 11 aa, Artificial Sequence
  Peptide
GRRAAPGRXGG

SEQ ID NO: 10 - DNA, 25 bp, Artificial Sequence
  Primer
gcctacatccctgtctagcagcatc

SEQ ID NO: 11 - DNA, 22 bp, Artificial Sequence
  Primer
cattctaaaacaaggatccaag

SEQ ID NO: 12 - DNA, 348 bp, Homo sapiens
atgacattttccagaggaacgtgtaaggaagtgccagaggcaaggagagcacctggaagcctacatccctgtctagcagcatcatgctcagctgctggcttgcacacaagctcgtggaagaacctgttttggatagaaggactagtaagtatttgcctagggcacatagttgtacaagagacggacgtttttaggtccttgcggtttcttgcatttccagaaaacttgcttcaaatatttttccagatgcaaaattccttggatccttgttttagaatgaatctattaactaaactggatccagaaaaagtctataatcagttttgtttttcagaaacttcacattaa

SEQ ID NO: 13 - protein, 115 aa, Homo sapiens
MTFSRGTCKEVPEARRAPGSLHPCLAASCSAAGLHTSSWKNLFWIEGLVSICLGHIVVQETDVFRSLRFLAFPENLLQIFFQMQNSLDPCFRMNLLTKLDPEKVYNQFCFSETSH

SEQ ID NO: 14 - protein, 19 aa, Artificial Sequence
  Peptide
KEVPEARRAPGSLHPCLAA

SEQ ID NO: 15 - DNA, 27 bp, Artificial
  Primer
agctgttggctgaaaaggtggtctatg

SEQ ID NO: 16 - DNA, 26 bp, Artificial Sequence
  Primer
gcgcttctggtggcccttggagtgtg